(19) United States
(12) Patent Application Publication
(10) Pub. No.: US 2004/0109845 A1
(43) Pub. Date: Jun. 10, 2004

(54) Method for inhibiting articular cartilage matrix calcification

(76) Inventor: Robert Terkeltaub, San Diego, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/669,540

(22) Filed: Sep. 23, 2003

Related U.S. Application Data
(63) Continuation-in-part of application No. PCT/US02/09009, filed on Mar. 23, 2002.
(60) Provisional application No. 60/278,511, filed on Mar. 23, 2001.

(51) Int. Cl. A61K 48/00

(52) U.S. Cl. 424/85.2; 514/44

(57) ABSTRACT

Methods of inhibiting calcification in meniscal and articular cartilage of the joints are disclosed. The methods include blocking the activation and activity of transglutaminases tTGase and Factor XIIIa. Furthermore, disclosed are methods for identifying agents that affect TGase activity and/or matrix calcification.

### RELATED APPLICATION DATA

This application is filed under 35 U.S.C. §120, as a continuation-in-part application of co-pending PCT Application Serial No. PCT/US02/09009, filed Mar. 23, 2002, which claims priority under 35 U.S.C. 119(e) to U.S. Provisional Application No. 60/278,511, filed Mar. 23, 2001, both of which are herein incorporated by reference in their entirety.

### STATEMENT OF GOVERNMENT SUPPORT

This work was made with government support under grants from the Department of Veterans Affairs, NIH (P01 AGO7996, AR40770, HL61731), Arthritis Foundation, and Medical Research Council of Canada. The United States government has certain rights in this invention.

### FIELD OF THE INVENTION

The present invention is directed to the prevention or therapy of chondrocalcinosis due to aging, osteoarthritis and the like. The invention is more particularly concerned with treatment or prevention of calcification in meniscal and articular cartilage of the joints by blocking the activation and activity of transglutaminases tTGase and Factor XIIIa.

### BACKGROUND INFORMATION

Calcification of the pericellular matrix is a prevalent finding in aging and osteoarthritic articular cartilages and meniscal fibrocartilages (1,2). Moreover, crystals of hydroxyapatite and calcium pyrophosphate dihydrate (CPPD) released from the cartilage matrix can activate resident intra-articular mononuclear leukocytes and synovial lining cells (1,2). Consequent crystal-induced inflammation and expression of connective tissue-degrading enzymes can contribute to further cartilage degradation in degenerative joint disease (1,2).

In contrast to the physiologic mineralizion that occurs in growth plate cartilage (3), articular cartilage does not normally calcify (1,2,4). Nevertheless, certain factors that modulate endochondral growth plate chondrocyte differentiation and mineralization also have the potential to modulate pathologic calcification of articular and meniscal cartilages (3). For example, PTHrP, a major mediator of temporal and spatial endochondral chondrocyte differentiation and matrix metabolism, is up-regulated in OA cartilage (5,6). In addition, sequential chondrocyte hypertrophy and apoptosis develop adjacent to the mineralizing front in the growth plate (3). Moreover, fibral chondrocyte differentiation to hypertrophy and increased chondrocyte apoptosis are common findings in osteoarthritic (OA) cartilage (7,8). Chondrocyte hypertrophy also is a frequent finding adjacent to articular cartilage deposits of CPPD crystals (9).

One of the features of growth plate chondrocyte differentiation proposed to promote matrix calcification is increased expression of certain TGases (EC 2.3.2.13) in the hypertrophic zone (10,11). The central effect of TGases is induction of post-translational protein cross-linking in cells and in extracellular matrices. In this calcium-dependent reaction, the gamma-carboxyamide group of a peptide-bound glutamine residue and the primary amino group of either a peptide bound lysine or a polyamine are covalently joined to form a γ-glutamyl-ε-lysine or polyamine bond (12,13).

It has been proposed that TGase-induced polymerization of pericellular skeletal matrix calcium-binding proteins stabilizes the matrix and promotes nucleation and/or growth of calcium-containing crystals (12-14). Skeletal matrix proteins with amine acceptor sites for TGases include collagens I and II, and fibronectin and a variety of calcium-binding proteins (12,14-15). But it also has been demonstrated that TGases have the capacity to modulate processes that may indirectly affect matrix calcification in chondrocytes, such as signal transduction, cell adhesion, and activation of latent TGF□ (16-19). TGases also modulate the apoptotic process (20-22), which is pro-mineralizing (23). In this context, increased TGase expression has been employed as a tissue marker of increased apoptosis (23,24).

Seven distinct forms of TGase have been identified, the most widely expressed of which is Tissue TGase (tTGase, or TG_{c} or type II TGase) (12,13). TGases with limited tissue distribution include epidermal, keratinocyte, osteoblast and prostatic TGases (12,13). A major circulating TGase is Factor XIII, a coagulation protein involved in clot stabilization (12,13,25). The plasma form of Factor XIII is a latent (zymogen), soluble heterotetramer consisting of two “a” subunits (containing the catalytic site) and two “b” protein subunits (25). Plasma Factor XIII zymogen requires thrombin for proteolytic activation to an active TGase (25). Importantly, a latent tissue form of Factor XIII (factor XIIIa) also has been identified (25). This form of Factor XIIIa, which consists only of two “a” subunits, is known to be expressed in not only platelets, monocytes, skin, placenta and gut, but also in growth plate cartilage (25).

In avian and non-avian skeletons, Factor XIIIa and tTGase expression have been observed to be temporally and spatially associated with terminal differentiation and matrix calcification in growth plate chondrocytes (10,11,26). TGases are generally regulated not only at the level of gene expression but also by a variety of cell activation and differentiation-associated post-translational changes that promote increased TGase enzymatic activity (12). For example, hypertrophic chick chondrocytes have been demonstrated to express intracellular thrombin-like proteolytic activity with the capacity to activate the Factor XIIIa zymogen (10).

Porcine articular chondrocytes have recently been observed to express tTGase, and porcine chondrocyte TGase enzymatic activity rises several-fold in aging (27). Moreover, porcine articular chondrocyte TGase activity was implicated in augmenting extracellular PPi(27), a major regulator of matrix calcification whose production by articular chondrocytes is TGF□-inducible and rises in association with aging (28). Thus, our objectives in this study were to explore TGase expression and activation in cells of human joint cartilages, to assess cartilage TGase activity in aging human joint cartilages, and to test the hypothesis that TGases directly promoted matrix calcification by chondrocytic cells. Clinical studies have linked cartilage calcification to worsening OA. The ability of specific TGases to directly stimulate matrix calcification provides novel molecular targets for potential prevention and control of chondrocalcinosis, which we will further dissect in this project. Because of the close linkage of OA and the multifaceted effects of TGases on cell function, the proposed studies also may lead to definition of novel mechanisms by which chondrocyte function becomes generally compromised in aging and OA.

OA and chondrocalcinosis are major public health problems that become particularly prevalent with aging. Therefore the patent is directly pertinent to cartilage diseases that afflict a large proportion of the patient population and will help in developing new and more effective therapeutic strategies.

In view of the above discussion, it is evident that there exists a strong need for an effective therapy for prevention and treatment of chondrocalcinosis of cartilage, including cartilage damaged as a result of injury and/or disease. There is also a continuing need to develop treatment methods that achieve therapeutic efficacy while minimizing toxicity and adverse events. The present invention attempts to fulfill these needs and provides additional advantages that will be apparent from the detailed description below.

### SUMMARY OF THE INVENTION

The present invention provides a method for suppressing pathological calcification of the meniscal and articular cartilage matrix. The method involves inhibiting at the level of activation and/or activity of the zymogen Factor XIIIa (FXIIIa) and tissue transglutaminase (tTGase) in chondrocytes. Inhibition of activation is accomplished by blocking the production of one or more of molecules selected from the group consisting essentially of interleukins IL-1, IL-8, nitric oxide donor Noc-12, peroxynitrite generator Sin-1, tumor necrosis factor a (TNFα), or S100 family of proteins. The inhibition of activation can also be accomplished by blocking TNFα receptor-associated signaling factors (TRAFs), TRAF2 and TRAF6.

Alternatively, the inhibition can be accomplished by expressing the finger protein A20 in chondrocytic cells.

In another embodiment, the present invention provides a method for preventing or treating chondrocalcinosis in aging and osteoarthritic (OA) cartilages by suppressing the ability of the articular cartilage matrix to be pathologically calcified. This is achieved by blocking activation of the enzyme tTGase and zymogen FXIIIα. The blocking is accomplished by removal, or down-regulation of activators such as IL-1, IL-8, Noc-12, Sin-1, TNFα, and S100 proteins.

The blocking is also accomplished by expressing the zinc finger protein (A20) in chondrocytes. A20 suppresses IL-1-induced nitric oxide production and inhibits both IL-1 and TNFα signaling partly at the level of TRAF2 and TRAF6 action by inhibiting NF-κB activation. The expressing is accomplished by transfection of chondrocytes wherein the transfection markedly upregulates meniscal cell production of A20. The up-regulation of A20 prevents or minimizes cartilage degradation and matrix calcification in vivo.

Further contemplated is a method for preventing or treating cartilage matrix calcification by suppressing extracellular cartilage Factor XIIIa and tTGase activity that promotes polymerization of secreted calcium-binding proteins, which in turn promotes extracellular calcium precipitation. The calcium-binding proteins are S-100, fibronectin and osteonectin.

In another embodiment, the present invention provides a method for inhibiting TGase activity of zymogen Factor XIIIa (FXIIIa) and/or tissue transglutaminase (tTGase) in a chondrocyte. The method includes contacting the chondrocyte with an effective amount of an inhibitor of IL-1, Noc-12, Sin-1, tumor necrosis factor a (TNFα), and/or TNFα receptor-associated signaling factor (TRAFs), TRAF2 and TRAF6.

In another embodiment, the present invention provides a method for identifying an agent that affects matrix calcification. The method includes contacting a chondrocyte in vitro with a test agent under conditions for inducing matrix calcification, wherein the chondrocyte expresses zymogen factor FXIIIa and/or tissue transglutaminase (tTGase). Next, the effect of the test agent on matrix calcification is determined, wherein an effect on matrix calcification identifies the test agent as an agent that affects matrix calcification. In certain aspect, the chondrocyte is transfected with a TGase expression vector for expressing zymogen factor FXIIIa or tTGase.

### BRIEF DESCRIPTION OF THE DRAWINGS

FIGS. 1A-1D show a comparison of Factor XIIIa and tTGase expression in normal and osteoarthritic (OA) knee articular cartilages and menisci. Normal human knees (obtained at autopsy) and OA knees (obtained at time of total knee replacement), as well as control normal tibial growth plates were sectioned and studied by immunohistochemistry using the avidin biotin alkaline phosphatase (ABC) method, as described in the Methods. Hematoxilin was used as the counterstain. Brown-red staining was considered to be positive by this method.

FIG. 1A: As a positive control in assessing chondrocytic Factor XIIIa and tTGase expression, tibial growth plates of human fetal tissue (160 days gestation) were studied, as described in the Methods (upper panels X100 and lower panels X400 magnification). The results show Factor XIIIa and tTGase staining in the hypertrophic zone, representative of studies with two donors. Here and elsewhere in the figures, NC indicates negative control.

FIG. 1B: Results are shown for normal adult articular knee cartilage specimens from one subject (X400, representative of studies with 4 different donors). Factor XIIIa and tTGase expression were both detected in superficial zone chondrocytes of normal articular cartilage and to a lesser degree in deep zone chondrocytes.

FIG. 1C: Results are shown for human OA knee articular cartilage from one subject, sampled at the time of total joint replacement for the disease (X400, representative of studies with 4 different donors). The Figure demonstrates the markedly up-regulated tTGase and Factor XIIIa expression by enlarged chondrocytes in both the superficial and deep zones of articular cartilage.

FIG. 1D: Results are shown for the central zone of the medial meniscus of a normal knee and the medial meniscus of an OA knee (sampled at time of total knee replacement for severe OA), each from an individual subject (X200, each representative of 4 different subjects). The results indicated trace Factor XIIIa and tTGase expression in the normal meniscus, and marked up-regulation of Factor XIIIa and tTGase expression in enlarged ceils in the OA meniscus.

FIGS. 2A-2B demonstrate effects of IL-1β and TGFβ on expression of Factor XIIIa and tTGase in knee articular cartilage and medial meniscus in organ culture. Articular cartilage and medial menisci from normal donors were studied in cartilage organ culture in media supplemented with 1% FCS and 1% L-glutamine, as described in the Methods, following 48 hours of treatment with TGFβ 10 ng/ml, IL-1β (1 ng/ml), or medium alone.

FIG. 2A: Knee articular cartilages were studied by immunohistochemistry for tTGase and Factor XIIIa expression, as described above (X100, with insets (X200) showing magnified areas in the superficial zone). The Figure from an individual subject (representative of studies with three donors) revealed that both IL-1 and TGFβ up-regulated tTGase expression. Factor XIIIa expression was up-regulated by IL-I and (to a lesser extent) by TGFβ.

FIG. 2B: The Figure shows the central zone of the medial meniscus from an individual subject (X200, representative of studies with three donors). The Figure reveals that TGFβ and IL-1β treatment induced increased tTGase and Factor XIIIa immunostaining.

FIGS. 3A-3B are a comparison of specific activities of tgase with the matrix calcification-regulatory enzymes NTPPPH and AP in menisci of various ages. To assess if TGase activity increased in association with cartilage aging, were studied samples taken from 50 mg dry weight from the central zone of the medial menisci of 38 donors of different ages. For the TGase assays, aliquots of 5 μg of soluble protein were prepared, as described in the Methods, and added aliquots in triplicate to a plate previously coated with 20 mg/ml of N,N-dimethylcasein. For the TGase assays (FIG. 3B), performed as described in the Methods, the results were expressed as total 5-(Biotinamido) pentylamine (BP) incorporated into N,N-dimethylcasein (nmol/μg cartilage DNA). For NTPPPH and AP assays (FIG. 3B), aliquots of 5 μg of soluble protein were assayed in triplicate, as described in the Methods. Results were analyzed by linear regression, with results indicated.

FIGS. 4A-4C show the association of increased TGase and NTPPPH but not AP specific activities with increased severity of OA in the meniscus of aged donors. Meniscal specimens (50 mg blocks from the central (chondrocytic) region of the medial meniscus) were taken at the time of autopsy or total joint arthroplasty for OA in a panel of 45 donors over the age of 60. This panel of donors was entirely separate from the panel of donors studied in FIG. 3. Cartilage samples were graded in a blinded manner for degree of OA as follows: Grade 1, intact cartilage surface; Grade 2, minimal fibrillation; Grade 3, overt fibrillation; Grade 4, erosion. TGase (FIG. 4A), NTPPPH (FIG. 4B), and AP (FIG. 4B) specific activities (per μg cartilage DNA) were then studied in a blinded manner, performed as described above. *p<0.05.

FIGS. 5A-5C show IL-1 selectively induced TGase activity (and not NTPPPH and AP activity) in meniscal cells in primary culture; association with aging. Meniscal cells (from the same donors used in FIG. 3 above) were released from the matrix with collagenase digestion, and grown in monolayer culture for 72 hrs, then trypsinized and plated in 35 mm dishes (containing 3×10⁵ cells) and allowed to adhere overnight. Cells were then treated for 48 h with 10 ng/ml of TGFβ or IL-1β (10 ng/ml) in 1% FCS-containing DMEM high glucose medium. The cells were collected and lysed for the TGase (FIG. 5A), NTPPPH (FIG. 5B), and AP (FIG. 5C) assays as described in the Methods. Five micrograms of soluble protein was studied in triplicate for each assay performed as previously described. Results were analyzed by linear regression, with results indicated.

FIGS. 6A-6B TGase activity induced by IL-1 in meniscal cells was attributable in part to both tTGase and FXIIIa Meniscal cells (3×10⁵) were cultured in 35 mm dishes for 72 hrs in 1% FCS containing DMEM high glucose media with or without IL-1β(10 ng/ml).

FIG. 6A. To assess for possible immunoprecipitatation of active tTGase and Factor XlIIa, 1 μl (0.1 μg) of antibody to FXIIIa or tTGase , or nonimmune IgG was added to equal aliquots (100 μg protein) of lysates of meniscal cells that had previously treated with IL-1, as described above. This was followed by pre-clearance of the cell lysates that had been pre-cleared with nonimmune IgG, as described in the Methods. The samples were mixed at 4° C. for 1 hr followed by the addition of Protein G Sepharose beads to a final ratio of 10% (v/v), followed by remixing for 1 h, centrifugation at 14,000×g for 1 min, and washing of beads and resuspension in a Tris HCI-containing buffer, pH 7.5, as described in the Methods, Protein precipitated was quantified for each sample, and then 5 μg aliquots were used for determinations of TGase activity, as above.

FIG. 6B. To assess for possible neutralization of TGase activity attributable to FXIIIa or tTGase, meniscal cell lysates (100 μg protein from cells that had been treated with IL-1, as above) were incubated with 0.1 μg of antibody to FXIIIa or tTGase, or nonimmune IgG as a control in an equal volume for 2 h at 4° C. The TGase activity of each cell extract was then determined as previously described. Data pooled from cell lysates of 3 normal donors studied in triplicate. *p<0.05.

FIG. 7 shows that peroxynitrite and NO donors, and TNFα, but not TGFβ, induce TGase activity in cultured meniscal cells. Meniscal cells (3×10⁵) were cultured in 35 mm dishes for 72 hrs in the presence of the indicated concentrations of the peroxynitrite donor Sin-1, the NO donor Noc- 12, TNFα and TGFβ. The cells were lysed and 5 μg of total protein was assayed for TGase activity as described previously. Data pooled from 5 normal donors studied in triplicate. *p<0.05.

FIGS. 8A-8B demonstrate that the zinc finger protein A20, like the NOS inhibitor NMMA, suppresses IL-1 and TNFα-induced NO release in meniscal cells. NO release by 3×10⁵ meniscal cells, which were plated and stimulated with TNFα (10 ng/ml) or IL-1 (10 ng/ml), with or without 10 μM NMMA (FIG. 8A), for 72 hrs was studied. Conditioned media were collected and 50 μl aliquots analyzed for NO release using the Greiss Reaction, with results expressed as μmoles released per μg DNA. For the studies in FIG. 8B, the meniscal cells were transfected with A20 or empty plasmid (where indicated), using the procedure described in the Methods, which included use of Lipofectamine Plus and a prior 3 minute incubation with 0.00015% digitonin to optimize the transfection. After 72 hrs of incubation with TNFα or IL-1, the conditioned media were collected from the cultured, transfected cells and analyzed in triplicate for NO release, as above. Data pooled from 4 normal donors studied in triplicate for FIGS. 8A and 8B. *p<0.05.

FIG. 9 shows that A20 suppresses the ability of IL-1 and TNFα to induce TGase activity in cultured meniscal cells. Aliquots of 3×10⁵ normal knee meniscal cells, which were transfected with empty vector or with the A20 expression plasmid were studied, as described above, plated in 35 mm dishes and allowed to adhere overnight. Cells were then incubated an additional 72 hrs in the presence of TNFα (10 ng/ml), IL-1 (10 ng/ml), Sin-1 (10 μM), Noc-12 (25 μM), where indicated. The cells were lysed and 5 μg aliquots of total protein were assayed for TGase activity as described previously. Data pooled from 4 donors studied in triplicate for both FIGS. 9A and 9B. *p <0.05.

FIG. 10 refers to effects of direct Factor XIIIa and tTGase expression on TGase activity in meniscal cells and TC28 cells. Human chondrocytic TC28 cells or normal knee meniscal cells (5×10⁵), were plated in 60 mm dishes and allowed to adhere overnight, as described in the Methods. Two μg of plasmid DNAs or empty plasmid vector were then transfected, performed as described above. After 24 hrs, the cells were removed from the dishes and plated on polyHEME-coated tissue culture plates and media supplemented with 10 mM P-glycerophosphate, ascorbatte (50 μg/ml) and dexamethasone (10⁻⁸ M), which stimulated the cells to form mineralizing nodules for 3-10 days in culture. For comparison purposes, cells were treated only with IL-1 or TGFβ (10 ng/ml each) for up to 10 days. At each time point designated, the cell nodules were collected and lysed, and TGase activity then determined as previously described. For meniscal cells, data pooled from 6 donors studied in triplicate. For TC28 cells, n=6 each, studied in triplicate. Statistics not indicated on this graph, but p<0.05 for the increases in TGase activities in response to both Factor XIIIa and tTGase transfection, and for IL-1 treatment, at all time points.

FIG. 11 shows effects of increased Factor XIIIa and tTGase-associated TGase activities on matrix calcification in TC28 cells and meniscal cells. TC28 cells and meniscal cells were transfected with empty plasmid or a TGase expression construct, and then transferred to polyHEME and cultured for up to 10 days, as described above. For comparison purposes, control untransfected cells were treated with IL-1 or TGFβ(10 ng/ml each) for up to 10 days. To measure matrix calcification, media and cells were removed from the PolyHEME-coated dishes, and 1 ml Alizarin Red S solution (0.5% v/v Alizarin Red S, pH 5.0) added to washed plates at 23° C. for 10 min. After further washing, 100 mM Cetylpyridium Chloride was added for 10 min to release the remaining calcium-bound Alizarin Red S. The solution was collected and read at OD₅₇₀, with 1.0 OD₅₇₀=1 unit of Alizarin Red released per μg of DNA per culture dish. n=6 each, studied in triplicate, and using 6 different knee meniscal donors. *p<0.05.

FIG. 12 illustrates that calgranulin S100A11 induces TGase activity in TC28 cells. TC28 cells were transfected with recombinant S100A11, S100A6, or control empty vector and TGase activity measured at 48 hours. n=6, p<0.05 for effects of S100A11. S100A11 is a cytokine-like ligand for the RAGE receptor, which is involved in chronic inflammatory and degenerative diseases including diabetic nephropathy and atherosclerosis. S100A11 is one of the likely factors involved in upregulating TGase activity in osteoarthritic cartilage.

FIG. 13 illustrates that S100 and tTGase (TG2) synergistically increase matrix calcification by chondrocytic cells. TC28 cells were transfected with recombinant calgranulin family members (S100A11 and S100A8) and/or treated with soluble tTGase(TG2) purchased from Sigma (St. Louis, Mo.) and added at the indicated concentrations for 48 hours following transfection. Calcification was measured a bound Alizarin Red S at 48 hours; n=8, *p<0.05 relative to control.

FIG. 14 illustrates that IL-1 and IGF-1 , which are involved in the pathogenesis of osteoarthritis, both induce mRNA expression of S100A11 in human articular chondrocytes. Normal human knee articular chondrocytes were treated in culture with IL-1 or IGF-I (10 ng/ml) for 24 hours and ascertained for S100A11 mRNA expression by RT-PCR, relative to the loading control L30, a ribosomal housekeeping gene. Results for induction of S100 A11 representative of 3 separate experiments with different donors.

FIG. 15 schematically illustrates the factors that are involved in osteoarthritis and chondrocalcinosis.

FIG. 16 diagrammatically illustrates a model for how Chondrocytes construct matrix calcification.

### DESCRIPTION OF THE PREFERRED EMBODIMENT

Calcification of the pericellular matrix by chondrocytes (chondrocalcinosis) is prevalent in aging and osteoarthritic (OA) cartilages, induces joint inflammation, and appears to worsen cartilage degeneration. Conversely, inflammation stimulates chondrocytes to calcify their matrix. Specifically, IL-1 induces expression of two distinct covalent protein crosslinking enzymes of the transglutaminase (TGase) family:tissue TGase (tTGase) and Factor XIIIa, the tissue form of the blood coagulation protein. IL-1 and nitric oxide donors also markedly up-regulates TGase enzymatic activity, an effect shared by TNFα.

Factor XIIIa is expressed as a latent enzyme (zymogen) that must be converted to an active form. In addition, the enzyme activity of all TGases, including tTGase and Factor XIIIa, is under tight physiologic regulation by factors including ambient calcium, post-translational proteolysis and phosphorylation, and, in the case of tTGase, GTP binding. Significantly, TGase activity increases in direct association with OA severity and aging in knee cartilages. TGase activity also increases in a striking age-dependent manner in chondrocytes isolated from human knee meniscal cartilage in response to IL-1. Active Factor XIIIa and tTGase both directly stimulate matrix calcification. Thus, marked increases in TGase activity in aging and OA cartilages, as well as other diseased tissues, appear physiologically significant.

Current Status of the Area

Deposition of CPPD and hydroxyapatite (HA) crystals in aging and OA cartilages is common and can significantly contribute to MMP and IL-1 expression, joint inflammation, and degradation of the cartilage matrix. Conversely, chondrocyte generation of IL-1 and certain inflammatory mediators implicated in OA pathogenesis stimulates matrix calcification. Recently, we demonstrated that IL-1 and TNFα stimulate TGase activity in chondrocytes in a NO-dependent-manner. Furthermore, we provided the first direct evidence that activated TGases stimulate matrix calcification. We now propose to develop TGase inhibition as a therapeutic strategy for calcification disorders.

In a calcium-dependent transamidation reaction (EC 2.3.2.13), TGases covalently link donor glutamine residues to acceptor primary amino groups of other proteins or of polyamines. TGases thereby catalyze covalent post-translational protein cross-linking in cells and in extracellular matrices. The matrix stabilization stimulated by TGases functions in processes including blood clot organization and the organization and healing of wounds and other inflammatory responses.

At least 8 distinct TGase isozymes are expressed in humans, including several TGases with limited tissue distribution, and alternatively spliced tissue-specific forms of the most widely expressed TGase, known as Type II or Tissue TGase (tTGase). However, our own TGase expression profiling, supported by additional literature, has indicated that articular chondrocytes express only two TGase isozymes: tTGase, and Factor XIIIa, which is the tissue-expressed form of coagulation Factor XIII.

Dysregulated activity of TGases has the potential to alter tissue function and architecture. Therefore, TGases are highly regulated at the level of expression, including induction by IL-1, TGFβ, and retinoic acid. But TGases also are regulated at the level of catalytic activity via subcellular modulation of calcium and by a variety of post-translational protein modifications influenced by cell activation and differentiation. Post-translational changes capable of increasing activity of TGases include proteolysis, phosphorylation, and lipidation (112).

Regulation of the tTGase isozyme is unique among the TGases, because it is a dual function enzyme (ATPase/GTPase and TGase). Specifically, GTP binding to an isozyme-specific ATP/GTP-binding domain in tTGase induces a conformational change that attenuates TGase catalytic activity. The binding of GTP to tTGase can be regulated by receptor-linked signaling. For example, the signaling molecule phosphatidylinositol (PI)-specific Phospholipase C (PLC) delta1 stimulates both binding of GTP to tTGase and inhibition of the GTPase activity of tTGase.

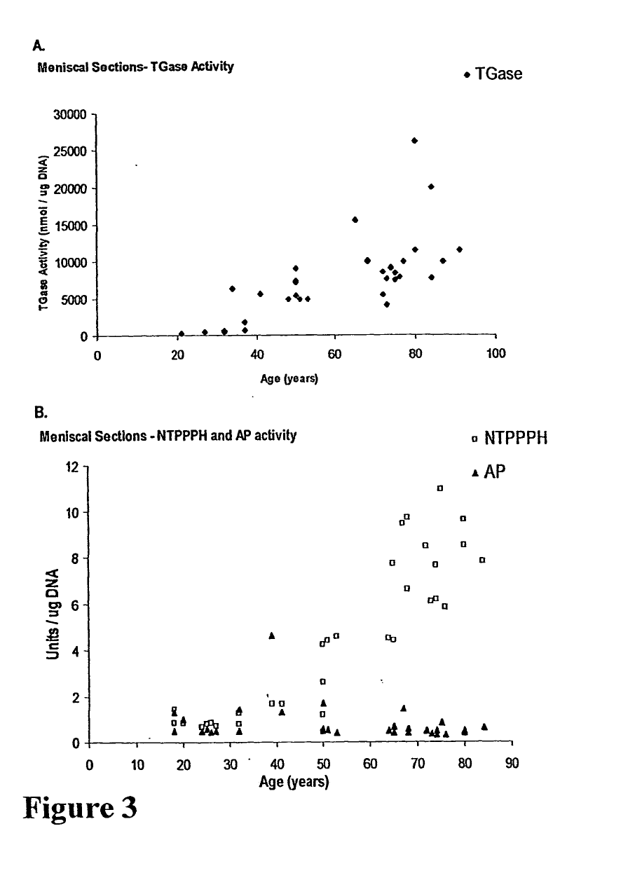

Certain activation mechanisms in chondrocytes of Factor XIIIa may be shared or distinct from those of the tTGase isozyme. Significantly, Factor XIIIa Plasma Factor XIII is a latent enzyme (zymogen): the heterotetramer composed of two “a” and two “b” subunits requires thrombin for proteolytis of the “a” subunit to convert it to an active TGase (25). The tissue-expressed form of Factor XIIIa, which consists only of two “a” subunits (FIG. 3), also is a latent TGase. Thrombin is sparse in the uninflamed joint. However, hypertrophic chick sternal chondrocytes have been demonstrated to express intracellular thrombin-like proteolytic activity with the capacity to activate the Factor XIIIa zymogen.

Activated TGases stimulate matrix calcification. Tissue forms of TGases are primarily cytosolic, though tTGase also can concentrate in specialized areas on the inner leaflet of the plasma membrane. In addition, tTGase and factor XIIIa can be partly extruded from cells (10,12), and the co-localization of tTGase with pericellular fibronectin appears to modulate matrix assembly. Skeletal matrix proteins with amine acceptor sites for TGases include major and minor collagens and a variety of calcium-binding proteins, such as S100 family members, osteonectin, and osteocalcin. Thus, it is likely that TGase-induced covalent stabilization of matrix calcium-binding proteins could directly enhance pericellular nucleation and/or growth of calcium-containing crystals in cartilage. Our data indicate that at least one of the S100 proteins, S100A9, not only enhances calcification by TGase but also may serve to activate TGase activity. Factor XIIIa and tTGase clearly have the potential to stimulate calcification by several other shared and TGase isozyme-selective functions discussed below.

How do TGases interface with central pathogenic mechanisms in cartilage calcification?:

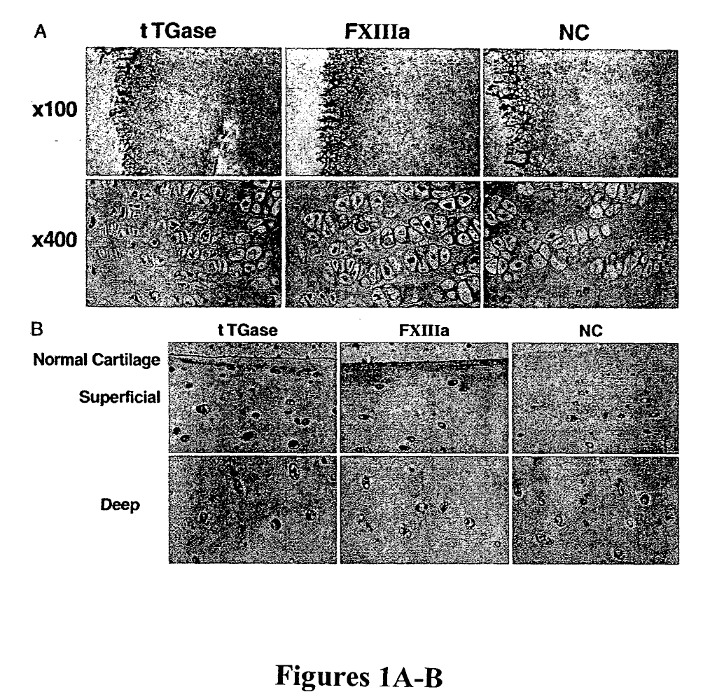

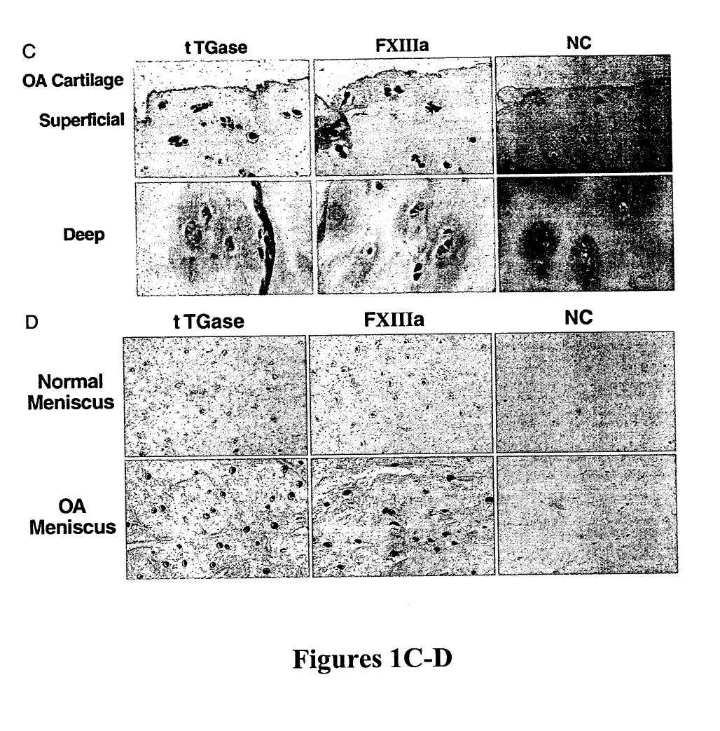

Clinically heterogeneous conditions promote cartilage calcification, including metabolic disorders (e.g., hyperparathyroidism) and familial forms of premature chondrocalcinosis. Correspondingly, the etiology of articular cartilage calcification is multifactorial. But the most prevalent forms of chondrocalcinosis, by far, are the idiopathic CPPD deposition disease associated with aging and the CPPD and HA crystal deposition intimately linked with OA. Major mechanisms implicated in the pathogenesis of these prevalent forms of cartilage calcification are schematized in FIG. 1. TGase activation and function appear to interface with each of these illustrated pathways.

First, matrix modification is a central feature of both OA and cartilage calcification. In this context, TGase activity increases in direct association with OA severity, and the potential for TGases to contribute to pro-mineralizing structural modification of the chondrocyte pericellular matrix was reviewed above.

Second, dysregulated chondrocyte ATP metabolism, including markedly heightened generation of inorganic pyrophosphate (PPi) from ATP by increased nucleotide pyrophosphophydrolase (NTPPPH) activity, is a central feature of idiopathic and OA-associated CPPD deposition disease. Cartilage PPi generation rises directly in association with aging, and it potently stimulated by TGFβ. The cartilage matrix may become saturated with not only PPi, but also with inorganic phosphate (Pi), generated by both PPi hydrolysis and by the effects of ATPases (FIG. 1). HA crystal deposition may thereby be promoted, often concurrent with CPPD deposition. Within this paradigm, TGase activation appears to participate by stimulating activating conformational changes in latent TGFP, thereby supporting chondrocyte PPi generation and facilitating other pathologic effects of TGFP including promotion of MMP-13 expression.

Third, a central process in OA is altered chondrocyte differentiation. Development of foci of chondrocyte hypertrophy and apoptosis in OA is significant partly because both of these alterations in differentiation promote calcification. Furthermore, CPPD and HA crystals are deposited in proximity to hypertrophic and apoptotic chondrocytes in articular cartilages. In both the hypertrophic zone of growth plate cartilage, and in hypertrophic chondrocytes in human articular cartilages, expression of the tTGase and Factor XIIIa isozymes both become markedly up-regulated in a co-localized manner. TGases promote extrusion of cytosolic contents and cell adhesion. Up-regulation of TGase activity may slow the apoptotic process (20-22). Furthermore, up-regulated TGase expression has been suggested to modulate differentiation of vascular smooth muscle cells. Thus, TGase up-regulation may help to alter the phenotype of the aging articular chondrocytes to a calcifying cell.

Last, TGase activity increases in direct association with aging in human knee cartilages. TGase activity also increases in a striking age-dependent manner in chondrocytes isolated from human knee meniscal cartilage in response to IL-1. In this context, IL-1 induces expression of tTGase and Factor XIIIa, in cartilage organ culture and IL-1 also markedly up-regulates TGase enzymatic activity in vitro, an effect shared by TNFα.

Accordingly, in one embodiment, the present invention provides a method for suppressing pathological calcification of the meniscal and articular cartilage matrix in a subject in need thereof, by contacting a chondrocyte of the subject with an effective amount of a TGase inhibitor that inhibits activation and/or activity of zymogen Factor XIIIa (FXIIIa) and/or tissue transglutaminase (tTGase) in the chondrocyte.

As used herein, an “effective amount” of an inhibitor is an amount that reduces tTGase and zymogen factor FXIIIa TGase activity of a chondrocyte in vivo or in vitro. It will be understood that using methods provided herein, and general methods known in the art, by trying various concentration ranges of inhibitors, an effective amount can be determined.

The methods of the present invention involve inhibiting at the level of activation and/or activity, zymogen Factor XIIIa (FXIIIa) and/or tissue transglutaminase (tTGase) in a chondrocyte. In certain aspects, inhibition of activation is accomplished by blocking production of IL-1, IL-8, nitric oxide donor Noc-12, peroxynitrite generator Sin-1, tumor necrosis factor a (TNFα), and/or a member of the S100 family of proteins. The inhibition of activation can also be accomplished by blocking TNFα receptor-associated signaling factors (TRAFs), TRAF2 and TRAF6.

In another embodiment, the inhibition is accomplished by expressing or increasing the expression of the zinc finger protein (A20) in chondrocytes. As discussed herein, A20 suppresses IL-1-induced NO production and the ability of IL-1 and TNFα to induce TGase activity. In certain illustrative examples, expression or increased expression of the A20 protein is accomplished by transfecting chondrocytes with an A20 expression vector. In certain aspects, the transfection markedly up-regulates meniscal cell production of A20. The upregulation in in vivo methods is sufficient to prevent or minimize cartilage degradation and matrix calcification in vivo.

In certain aspects the TGase inhibitor can act directly by inhibiting expression of tTGase or FXIIIa by Chondrocytes. For example, the inhibitor can be a polynucleotide agent that is, or that encodes, an antisense molecule, a ribozyme or a triplexing agent. For example, the polynucleotide can be (or can encode) an antisense nucleotide sequence such as an antisense tTGase or FXIIIa nucleotide sequence, which can act as an agonist to reduce or inhibit tTGase or FXIIIa expression. Such polynucleotides can be contacted directly with a target cell such as a chondrocyte and, upon uptake by the cell, can effect their antisense, ribozyme or triplexing activity; or can be encoded by a polynucleotide that is introduced into a cell, whereupon the polynucleotide is expressed to produce, for example, an antisense RNA molecule or ribozyme, which effects its activity.

An antisense polynucleotide, ribozyme or triplexing agent is complementary to a target sequence, which can be a DNA or RNA sequence, for example, messenger RNA, and can be a coding sequence, a nucleotide sequence comprising an intron-exon junction, a regulatory sequence such as a Shine-Delgarno sequence, or the like. The degree of complementarity is such that the polynucleotide, for example, an antisense polynucleotide, can interact specifically with the target sequence in a cell. Depending on the total length of the antisense or other polynucleotide, one or a few mismatches with respect to the target sequence can be tolerated without losing the specificity of the polynucleotide for its target sequence. Thus, few if any mismatches would be tolerated in an antisense molecule consisting, for example, of 20 nucleotides, whereas several mismatches will not affect the hybridization efficiency of an antisense molecule that is complementary, for example, to the full length of a target mRNA encoding a cellular polypeptide. The number of mismatches that can be tolerated can be estimated, for example, using well known formulas for determining hybridization kinetics (see Sambrook et al., supra, 1989) or can be determined empirically using methods as disclosed herein or otherwise known in the art, particularly by determining that the presence of the antisense polynucleotide, ribozyme, or triplexing agent in a cell decreases the level of the target sequence or the expression of a polypeptide encoded by the target sequence in the cell.

A polynucleotide useful as an antisense molecule, a ribozyme or a triplexing agent can inhibit translation or cleave the nucleic acid molecule, thereby inhibiting tTGase or FXIIIa expression or activity of a cell. An antisense molecule, for example, can bind to an mRNA to form a double stranded molecule that cannot be translated in a cell. Antisense oligonucleotides of at least about 15 to 25 nucleotides are preferred since they are easily synthesized and can hybridize specifically with a target sequence, although longer antisense molecules can be expressed from a polynucleotide introduced into the target cell. Specific nucleotide sequences useful as antisense molecules can be identified using well known methods, for example, gene walking methods (see, for example, Seimiya et al., J. Biol. Chem. 272:4631-4636 (1997), which is incorporated herein by reference). Where the antisense molecule is contacted directly with a target cell, it can be operatively associated with a chemically reactive group such as iron-linked EDTA, which cleaves a target RNA at the site of hybridization. A triplexing agent, in comparison, can stall transcription (Maher et al., Antisense Res. Devel. 1:227 (1991); Helene, Anticancer Drug Design 6:569 (1991)). Thus, a triplexing agent can be designed to recognize, for example, a sequence of a tTGase or FXIIIa gene regulatory element, thereby reducing or inhibiting the expression of a tTGase or FXIIIa polypeptide in the cell, thereby modulating TGase activity of a target cell, typically a chondrocyte.

In certain embodiments, expression and/or activity of tTGase and/or FXIIIa can be inhibited directly or indirectly using RNA interference (RNAi). For example, RNAi can be induced using short interfering RNAs (siRNAs), which are double-stranded RNAs, for example of 21-25 nucleotides in length, that function as key intermediaries in triggering sequence-specific RNA degradation during posttranscriptional gene silencing, as known in the art. siRNAs have a characteristic structure, with 5′-phosphate/3′-hydroxyl ends and a 2-base 3′ overhang on each strand of the duplex. siRNAs have been shown to induce gene-specific inhibition of expression in mammalian cells (Caplen, Natasha, J., et al., Proc. Natl. Acad. Sci., 98: 9742-9747 (2001). In certain aspects of the present invention, siRNAs that recognize a portion of the tTGase or FXIIIa gene are used as inhibitors. Alternatively, larger double stranded RNA polynucleotides (e.g. 100 to 1000 bp) can be used, which can be broken down by a cell into small double stranded RNA fragments that can trigger RNA interference.

The subject can be, for example, a mammalian subject such as a primate or a rodent, such as a mouse or a rat. In certain aspects the subject is a human. The subject can be afflicted with osteoarthritis, or from a line of an organism that is known to have an increased propensity to develop osteoarthritis and/or matrix calcification, or a similar animal disorder. For example the subject can be a human suffering from osteoarthritis, or a human at increased risk of developing osteoarthritis. Accordingly, the chondrocyte for methods of the invention can be for example, from a mammal such as a primate or a rodent, such as a mouse or a rat. In certain aspects the chondrocyte is a human chondrocyte.

The inhibitor of the methods of the invention can be delivered orally, intravenously, intraperitoneally, intramuscularly, subcutaneously, intranasally, and intradermally, as well as, by transdermal delivery (e.g., with a lipid-soluble carrier in a skin patch placed on skin), or even by gastrointestinal delivery (e.g., with a capsule or tablet). Furthermore, inhibitors used in the methods of the present invention in certain aspects are delivered directly to a site of chondrocyte matrix calcification., such as for example, directly to a joint, such as an arthritic joint. The dosage will be sufficient to provide an effective amount of an inhibitor either singly or in combination, as discussed above. Some variation in dosage will necessarily occur depending upon the condition of the patient being treated, and the physician will, in any event, determine the appropriate dose for the individual patient. The dose will depend, among other things, on the body weight, physiology, and chosen administration regimen.

The inhibitors employed in methods of the invention are administered alone or in combination with pharmaceutically acceptable carriers, in either single or multiple doses. Suitable pharmaceutical carriers include inert solid diluents or fillers, sterile aqueous solutions, and various nontoxic organic solvents. The pharmaceutical compositions formed by combining one or more inhibitors with the pharmaceutically acceptable carrier are then readily administered in a variety of dosage forms such as tablets, lozenges, syrups, injectable solutions, and the like. These pharmaceutical carriers can, if desired, contain additional ingredients such as flavorings, binders, excipients, and the like. Thus, for purposes of oral administration, tablets containing various excipients such as sodium citrate, calcium carbonate, and calcium phosphate are employed along with various disintegrants such as starch, and preferably potato or tapioca starch, alginic acid, and certain complex silicates, together with binding agents such as polyvinylpyrolidone, sucrose, gelatin, and acacia. Additionally, lubricating agents, such as magnesium stearate, sodium lauryl sulfate, and talc are often useful for tableting purposes. Solid compositions of a similar type may also be employed as fillers in salt and hard-filled gelatin capsules. Preferred materials for this purpose include lactose or milk sugar and high molecular weight polyethylene glycols.

When aqueous suspensions of elixirs are desired for oral administration, the inhibitors may be combined with various sweetening or flavoring agents, colored matter or dyes, and if desired, emulsifying or suspending agents, together with diluents such as water, ethanol, propylene glycol, glycerin, and combinations thereof. For parenteral administration, solutions of preparation in sesame or peanut oil or in aqueous polypropylene glycol are employed, as well as sterile aqueous saline solutions of the corresponding water soluble pharmaceutically acceptable metal salts previously described. Such an aqueous solution should be suitably buffered if necessary and the liquid diluent first rendered isotonic with sufficient saline or glucose. These particular aqueous solutions are especially suitable for intravenous, intramuscular, subcutaneous, and intraperitoneal injection. The sterile aqueous media employed are all readily obtainable by standard techniques well known to those skilled in the art.

An expression vector used in the methods of the present invention, typically includes, a polynucleotide encoding tTGase and/or FXIIIa, or, in other embodiments, A20, as well as regulatory elements useful for expressing the polynucleotide in a chondrocyte or chondrocyte-derived cell line. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther 1:51-64, 1994; Flotte, J. Bioenerg. Biomemb. 25:37-42, 1993; Kirshenbaum et al., J. Clin. Invest. 92:381-387, 1993; each of which is incorporated herein by reference).

In another embodiment, the present invention provides a method for blocking activation and/or activity of zymogen factor (FXIIIa) and tissue transglutaminase (tTGase) in a chondrocyte by contacting the chondrocyte with an effective amount of a transglutaminase inhibitor. In a related embodiment, the present invention provides a method for inhibiting TGase activity of zymogen Factor XIIIa (FXIIIa) and/or tissue transglutaminase (tTGase) in a chondrocyte, by contacting the chondrocyte with an effective amount of an inhibitor of IL-1, Noc-12, Sin-1, tumor necrosis factor a (TNFα), and/or TNFα receptor-associated signaling factor (TRAFs), TRAF2 and TRAF6. The methods of these embodiments, can be performed in vitro or in vivo.

In another embodiment, the present invention provides a method of identifying an agent that affects TGase activity of a chondrocyte. In certain aspects, the method identifies an agent that affects activation and/or activity of zymogen factor (FXIIIa) and tissue transglutaminase (tTGase) in a chondrocyte. Methods of this aspect of the invention are typically performed in vitro.

In another embodiment, the present invention provides a method for identifying an agent that affects matrix calcification by culturing a chondrocyte under conditions for inducing matrix calcification, contacting a chondrocyte in vitro with an effective amount of an inhibitor of activation and/or activity of zymogen factor FXIIIa and/or tissue transglutaminase (tTGase), and determining whether the test agent affects matrix calcification. In one aspect, the chondrocyte is transfected with a TGase expression vector before contacting the cell with the inhibitor.

As illustrated in the Example provided herein, matrix calcification can be measured in vitro with a quantitative Alizarin Red-binding assay. Conditions for inducing matrix calcification, for example include removing cultured Chondrocytes, or a cell line derived therefrom, and removing from their culture vessel, such as by contacting the cells with 0.2 mg/ml ethylenediaminetetraacetic acid (EDTA) and transferring the cells to a vessel coated with Poly (2-hydroxyethyl methacrylate) (polyHEME). The cells are then cultured in tissue culture medium that can be supplemented with an effective amount of beta-glycerophosphate, ascorbic acid, and dexamethasone (e.g., 10 mmol/L beta-glycerophosphate, 50 μg/ml ascorbic acid, and 10⁻⁸ mol/L dexamethasone). The culture medium, for example is DMEM high glucose (for meniscal Chondrocytes) or complete DMEM/F12 (for TC28 cells). After a sufficient time in culture, for example 10 days of culture, an Alizarin Red S binding assay can be performed, as disclosed in the Example herein.

In another embodiment, the present invention provides a method for identifying an agent that affects induction of matrix calcification. The method is performed by contacting the chondrocyte in vitro with an effective amount of an agent that activates and/or increases activity of zymogen factor FXIIIa and/or tissue transglutaminase (tTGase), and contacting the chondrocyte with a test agent. Matrix calcification of the chondrocyte is then determined and compared to matrix calcificatoin of the chondrocyte cultured under identical conditions, but without contact with the agent. A difference in matrix calcification between the chondrocyte contacted with the agent and the chondrocyte not contacted with the test agent, identifies the test agent that affects induction of matrix calcification. The agent that activates and/or increases activity of zymogen factor FXIIIa and/or tissue transglutaminase (tTGase) includes, for example, IL-1, Noc-12, Sin-1, or tumor necrosis factor a (TNFα).

In another embodiment, the present invention provides a method for identifying an agent that affects induction of chondrocyte TGase activity, comprising

contacting a test chondrocyte in vitro with an effective amount of an agent that activates and/or increases activity of a chondrocyte TGase;

contacting the test chondrocyte with a test agent;

contacting a control chondrocyte in vitro with an effective amount of an agent that activates and/or increases activity of a chondrocyte TGase;

determining TGase activity of the test chondrocyte and the control chondrocyte; and

comparing the TGase activity of the test chondrocyte and the control chondrocyte, wherein a difference in TGase activity between the test chondrocyte and the control chondrocytes, identifies the test agent as an agent that affects induction of chondrocyte TGase activity.

In certain aspects, the chondrocyte TGase is zymogen factor XIIIa (FXIIIa) and/or tissue transglutaminase (tTGase).

In another embodiment, the present invention provides a method for identifying an agent that affects induction of chondrocyte TGase activity. The method is performed by contacting the chondrocyte in vitro with an effective amount of an agent that activates and/or increases activity of zymogen factor FXIIIa and/or tissue transglutaminase (tTGase), and contacting the chondrocyte with a test agent. TGase activity of the chondrocyte is then determined and compared to TGase activity of the chondrocyte cultured under identical conditions, but without contact with the agent. A difference in TGase activity between the chondrocyte contacted with the agent and the chondrocyte not contacted with the test agent, identifies the test agent that affects induction of chondrocyte TGase activity. The agent that activates and/or increases activity of zymogen factor FXIIIa and/or tissue transglutaminase (tTGase) includes, for example, IL-1, Noc-12, Sin-1, or tumor necrosis factor a (TNFα).

For methods of the present invention performed in vitro, the chondrocyte can be, for example, a cultured chondrocyte, such as a cultured knee meniscal chondrocyte, or a chondrocyte-derived cell line, such as a TC28 cell.

A test agent for embodiments directed at identifying an agent that affects induction of chondrocyte TGase activity or induction of matrix calcification, can be, for example, an agent known to inhibit IL-1, Noc-12, Sin-1, or tumor necrosis factor a (TNFα). In certain aspects, the test agent is an agent known or suspected to inhibit IL-1 induced NO production. In certain aspects, the test agent is known or suspected to be a NOS inhibitor.

The term “test agent” is used herein to mean any agent that is being examined for the ability to affect TGase activity and/or matrix calcification. The methods of the invention involving a test agent can be used as a screening assay to identify molecules that can act as a therapeutic agent for treating osteoarthritis or to identify targets for osteoarthritis pharmaceuticals.

A test agent can be any type of molecule, including, for example, a peptide, a polypeptide, a protein, a peptidomimetic, a polynucleotide, or a small organic molecule, that one wishes to examine for the ability to act as a therapeutic agent, which is an agent that provides a therapeutic advantage to a subject receiving it.

Embodiments of the present invention directed at identifying an agent, for example can be performed as screening methods that provide the advantage that they can be adapted to high throughput analysis and, therefore, can be used to screen combinatorial libraries of test agents. Methods for preparing a combinatorial library of molecules that can be tested for a desired activity are well known in the art and include, for example, methods of making a phage display library of peptides, which can be constrained peptides (see, for example, U.S. Pat. Nos. 5,622,699; 5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O=Connell et al., supra, 1996; Tuerk and Gold, supra, 1990; Gold et al., supra, 1995; each of which is incorporated herein by reference); an oligosaccharide library (York et al., Carb. Res., 285:99-128, 1996; Liang et al., Science, 274:1520-1522, 1996; Ding et al., Adv. Expt. Med. Biol., 376:261-269, 1995; each of which is incorporated herein by reference); a lipoprotein library (de Kruif et al., FEBS Lett., 399:232-236, 1996, which is incorporated herein by reference); a glycoprotein or glycolipid library (Karaoglu et al., J. Cell Biol., 130:567-577, 1995, which is incorporated herein by reference); or a chemical library containing, for example, drugs or other pharmaceutical agents (Gordon et al., J. Med. Chem., 37:1385-1401, 1994; Ecker and Crooke, Bio/Technology, 13:351-360, 1995; each of which is incorporated herein by reference). Polynucleotides can be particularly useful as agents that can modulate a specific interaction of myostatin and its receptor because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

The invention will now be described in greater detail by reference to the following non-limiting examples.

### EXAMPLE 1

### Interleukin-1 Induces Pro-mineralizing Activity of Cartilage Tissue Transglutaminase and Factor XIIIA

This example illustrates that tTGase and Factor XIIIa activiy are increased in aging and degenerative cartilages and are increased by IL-1, nitric oxide donors, and tumor necrosis factor-alpha. Furthermore, the present example illustrates that TGase activity promotes matrix calcification cultured chondrocytes.

Methods

Reagents and Antibodies

Human recombinant TGF-β1 and IL-1β were purchased from R&D Systems (Minneapolis, Minn.). Rabbit polyclonal antibody to placental Factor XIIIa was from Calbiochem (La Jolla, Calif.), and goat polyclonal antibody to tTGase was obtained from Upstate Biotechnology (Lake Placid, N.Y.). Murine monoclonal anti-A20 antibody was a gift from Dr. C. Vincenz (Dept. of Pathology, U. of Michigan, Ann Arbor, Mich.) (29). All chemical reagents were obtained from Sigma (St. Louis, Mo.) unless otherwise indicated.

Meniscal Sections and Immunohistochemistry

Specimens of normal and degenerative articular cartilages and menisci were taken as full thickness blocks (approximately 1 mm in width and 2.5 mm in length) at autopsy or at the time of total knee replacement for advanced OA, as described previously and according to an institutionally approved protocol with appropriate informed consent (5,30). In the case of meniscal samples, we studied the central (chondrocytic) region (31) of the medial meniscus, and 5 micron paraffin-embedded sections were evaluated. Meniscal specimens were blindly graded for the severity of OA as follows: Grade 1, intact cartilage surface; Grade 2, minimal fibrillation; Grade 3, overt fibrillation; Grade 4; cartilage erosion (32).

Control specimens for normal human fetal growth plate tissue (160 days gestation) were obtained from the University of Washington Tissue Bank via an institutionally approved protocol. The whole knee was removed and fixed in 10% neutral buffered formalin. The nondecalcified tissues were embedded in paraffin and serial 5 micron sections were cut by microtome.

Immunohistochemistry was performed as previously described in detail (30,33,34). In brief, immunohistochemical sections (5 microns) were pretreated with bovine testicular hyaluronidase (0.5 mg/ml at 37° C. for 30 min) and incubated in 5% normal goat or rabbit serum for 20 min prior to avidin/biotin peroxidase staining by the ABC method. Hematoxilin was used as the counterstain. Biotinylated anti-rabbit or anti-goat antibodies served as secondary antibodies. Levamisole was added to block endogenous alkaline phosphatase (AP). Negative controls were nonimmune rabbit or goat serum as a substitute for primary antibody.

Meniscal Cell Isolation and Culture

Meniscal cells were taken from tissue slices removed from the central regions of the medial and lateral menisci. Where indicated, meniscal organ culture was performed by incubating these slices for b 48 hours in DMEM high glucose containing 1% FCS and 1% L-glutamine (and articular cartilage organ culture performed in the same manner, where indicated). Otherwise, we minced meniscal tissue with a scalpel, incubated in DMEM high glucose containing 2 mg/ml clostridial collagenase, 5% FCS, 1% L-glutamine, 100 units/ml Penicillin and 50 μg/ml Streptomycin (Omega Scientific, Tarzana, Calif.), and incubated on a gyratory shaker at 37° C. until the tissue fragments were digested. Residual multicellular aggregates were removed by sedimentation (1,000×g), and cells were washed three times in DMEM containing 5% FCS.

Meniscal cells were maintained in DMEM high glucose and supplemented with 10% FCS, 1% L-glutamine, 100 units/ml Penicillin and 50 μg/ml Streptomycin (Omega Scientific, Tarzana, Calif.) and cultured at 37° C. with 5% CO₂. In monolayer cell culture studies involving stimulation by TGFE□ or IL-1, the cells were placed in DMEM high glucose containing 1% FCS, 1% L-glutamine, 100 units/ml Penicillin and 50 μg/ml Streptomycin. In all other studies, the cells were cultured in complete medium (as described above). Only primary or first passage meniscal cells were studied. Type II collagen and aggrecan expression were confirmed in each meniscal cell preparation using RT-PCR, as previously described, and employing G3PDH as a control (28).

TC28 Cell Culture

Human immortalized juvenile rib chondrocyte cells (the TC28 cell line originally from Dr. M. Goldring, Harvard Medical School, Cambridge, Mass.) were maintained in DMEM/F12 (1:1) supplemented with 10% FCS, 1% L-glutamine, 100 units/ml Penicillin, and 50 μg/ml Streptomycin (Omega Scientific), and cultured at 37° C. with 5% CO₂ (28). Only TC28 cells between passages 25-45 were employed.

TGase Activity

TGase activity was measured by modifications to a previously described method (35). Specifically, we coated 96 well ImmunoModule plates (Nunc, Rochester, N.Y.) with 200 μl of 20 mg/ml N,N-dimethylcasein for 1 hr at 23° C. The N,N-dimethylcasein was removed and nonspecific protein binding was blocked by adding 3% BSA in 100 mM Tris, pH 8.5, 150 mM NaCl, 0.05% Tween-20 (TBST) to each well for an additional 1 hr at 23° C. Then, aliquots of 5 μg of total cellular protein from meniscal or TC28 cells that had been lysed and sonicated (in 5 mM Tris HCl, 0.25 M sucrose, 0.2 mM MgSO₄, 2 mM DTT, 0.4 mM PMSF, 0.4% Triton X100, pH 7.5), were added to the plate in triplicate. To measure TGase in extracts of whole menisci, 50 mg dry weight of tissue from the central zone of each medial meniscus was used as the source (after solubilization and sonication) for aliquots of 5 μg of soluble protein. Fifty μl of solution A (100 mM Tris, pH 8.5 and 20 mM CaCl₂) was added to all samples for TGase assay, followed by the addition of 50 μl of solution B (100 mM Tris, pH 8.5, 40 mM DTT and freshly added 2 mM 5-(Biotinamido) pentylamine (BP)). The plates were incubated for 1 hr at 37° C. The wells were washed once with TBST containing 1 mM EDTA and then three times with TBST. One hundred μl of a 1:500 dilution of streptavidin-AP in 3% BSA/TBST was added to each well for 1 hr at 23° C. The wells were washed twice with TBST, and 200 μl of solution C (100 mM Tris, pH 9.8, mM NaCl, 5 mM MgCl₂, 1 mg/ml of freshly added p-nitrophenylphosphate) was added to each well. Readings at OD₄₁₀ were taken over 15 minutes. Purified guinea pig liver TGase (Sigma) was used to prepare a standard curve. TGase activity was designated as the amount of BP incorporated into casein (per μg cellular DNA, determined chromogenically following precipitation in perchlorate (28), or, where indicated, per μg protein, determined as previously described (28)).

Transfection Studies and Culture of Nonadherent Meniscal Cells and TC28 Cells to Measure Mineralizing Conditions

For transfection of meniscal cells, we used recombinant human Factor XIIIa, a gift from Dr. Dominic Chong (U. of Washington, Seattle, Wash.). The cDNA insert was a 2.3 kb internal PstI fragment of full length cDNA containing 19 bp of the 5′ non-coding sequence, the entire coding region, and 140 bp of 3′ non-coding sequence, all cloned into the PstI site of pUC18. A human 3.3 kb full length tTGase cDNA construct, cloned into the EcoRI site of pSG5 was a gift of Dr. Peter Davies (U. of Texas, Houston, Tex.).

To directly induce expression of each TGase in meniscal cells, 5×10⁵ primary cells were plated in 60 mm dishes and allowed to adhere overnight. We modified (28) the manufacturer's protocol for the Lipofectamine Plus kit (Life Technologies, Grand Island, N.Y.). To optimize the transfection of meniscal cells, we added 2.0 ml of serum-free DMEM/F12 containing 0.00015% digitonin to washed cells and incubated for 3 min at 23° C. Then, media was removed, and cells transfected at 37° C. for 7 hrs, followed by removal of the media and addition of complete DMEM high glucose medium. Transfection of each TGase into TC28 cells was done by the same procedure, with the exception that the digitonin permeabilization step was omitted. For A20 transfection studies, we employed full length human A20 cDNA (a gift of Dr. M. Jaattela, Danish Cancer Society Research Center, Copenhagen, Denmark) (36) subcloned in sense orientation into the XhoI site of pcDNA4/HisMax (Invitrogen, Carlsbad, Calif.). Transfection efficiency, estimated by control transfections of β-galactosidase and staining for β-galactosidase, was consistently >40% for meniscal cells and >50% for TC28 cells.

To promote matrix calcification in short-term cultures, we modified a nonadherent chondrocyte culture system (37), and assessed cells that formed calcifying nodules over 10 days in culture. In brief, meniscal and TC28 cells, at 24 hours after the transfection, were washed and removed from the dish using 0.2 mg/ml EDTA, pH 8.0, then transferred to 6 well plates that had been previously coated with a 10% (wt./v) in 95% ethanol solution of Poly (2-Hydroxyethyl methacrylate) (polyHEME). Cells were then carried in complete DMEM high glucose (for meniscal cells) or complete DMEM/F12 (for TC28 cells) supplemented with 10 mM β-Glycerophosphate, 50 μg/ml Ascorbic Acid, and 10⁻⁸ M Dexamethasone. Cells were cultured for 10 days in these conditions, with media replaced every three days.

### Assessment of Matrix Calcification

To assay calcification of the pericellular matrix of meniscal cells and TC28 cells, we used a quantitative Alizarin Red S binding assay (38). In brief, the media and cells were removed from the PolyHEME coated dishes and the plates washed four times with PBS, followed by addition of one ml of 0.5% v/v Alizarin Red S, pH 5.0 at 23° C. for 10 min. The plates then were washed 4 times with PBS before the addition of 100 mM Cetylpyridium Chloride for 10 min to release the remaining calcium-bound Alizarin Red. The solution was collected and read at OD₅₇₀ on a SpectraMAX microplate reader (Molecular Devices, Sunnyvale, Calif.), with 1 OD₅₇₀=1 unit of Alizarin Red released per μg of DNA per culture dish. We extracted matrix crystals from plates under each condition using a papain-hypochlorite method (39), and the crystals were embedded in Spurr epoxy resin, sectioned, and viewed on a Philips EM340 transmission electron micrograph and analyzed by electron diffraction (40,41).

Assays of PPi metabolism, cellular DNA and NO

PPi was determined by differential adsorption on activated charcoal of UDP-D-[6-³H] glucose (Amersham, Chicago, Ill.) from the reaction product 6-phospho [6-³H] gluconate (28). Units of NTPPPH and AP were designated as micromoles of substrate hydrolyzed per hour (per μg DNA in each sample) (28). NO release by cultured meniscal cells was measured using the Greiss reaction (33). Concentrations or specific activities of PPi, NTPPPH, and AP were equalized for cellular DNA concentrations in each well.

Western Blotting and Immunoprecipitation Studies

SDS-PAGE and Western blotting were performed as previously described in detail (28), using the antibodies to FXIIIa, tTGase, and A20 cited above. To immunoprecipitate tTGase and Factor XIIIa from meniscal cells, 100 □g aliquots of cell lysates were precleared with 1% of Protein G Sepharose beads (from Sigma). One microliter (0.1 μg) of each antibody (FXIIIa, tTGase, and nonimmune controls) was added to the pre-cleared extract. The samples were mixed at 4° C. for 1 hr followed by the addition of Protein G Sepharose beads to a final volume/volume ratio of 10% . The tubes were again mixed for 1 hr and then centrifuged at 14,000 X g for 1 min. The beads were washed three times with PBS and resuspended in lysis buffer (5 mM Tris HCl (pH 7.5), 0.25 M sucrose, 0.2 mM MgSO₄, 2 mM DTT, 0.4 mM PMSF, 0.4% Triton X-100). The total protein precipitated was quantified for each sample. Then, 5 μg aliquots were used for determinations of TGase activity, as above.

### Caspase Activation Assays and TUNEL Staining of Cultured Cells

Caspase-1 and -3 activity was determined using the fluorescent substrates provided in the Promega (Madison, Wis.) Caspase Detection Kit according to the manufacturer's instructions. In brief, cell lysates were incubated for 1 hr at 37° C. in the provided buffer and then an additional 30 minutes with the substrates. Samples were analyzed at absorbance 360 nm, emission 410 nm. For TUNEL staining, 3×10⁵ cells were fixed with fresh 4% paraformaldehyde for 30 min at 23° C. Cells were permeabilized with 0.1% Triton X-100 and 0.1% sodium citrate for 2 min on ice and then washed twice with PBS. The DeadEnd Colorimetric Apoptosis Detection System (Promega, Madison, Wis.) was used to stain the cells (n≧200 for each experiment), according to the manufacturer's instructions.

Statistics

Error bars represent SD. Statistical analyses were performed using the Student's t test (paired 2-sample testing for means), and by analyzing correlation coefficients in linear regression studies, where indicated.

### Results

Expression and Localization of Factor XIIIa and tTGase in Normal and OA Human Knee Cartilages

To assess Factor XIIIa and tTGase expression and localization in joint cartilages, we used an immunohistochemical approach that first confirmed physiologic upregulation of chondrocyte expression of Factor XIIIa and tTGase (11) in the hypertrophic zone of epiphyseal cartilage (FIG. 1A). In normal knee articular cartilages, we detected some expression of Factor XIIIa and tTGase in flattened cells in the superficial zone, and trace expression of both TGases in the deep zone (FIG. 1B). The articular cartilages of human knees with severe OA (sampled at the time of total joint replacement) demonstrated markedly up-regulated tTGase and Factor XIIIa expression by enlarged chondrocytes in the superficial and deep zones of articular cartilage (FIG. 1C).

Similar to the findings in hyaline articular cartilages, trace expression of Factor XIIIa and tTGase was detectable in the central (chondrocytic) zone of knee medial menisci (FIG. 1D). Moreover, increased expression of both TGases was observed in enlarged cells in the central zones of medial meniscal cartilage sampled from severe OA, in specimens taken at the time of total knee joint replacement (FIG. 1D).

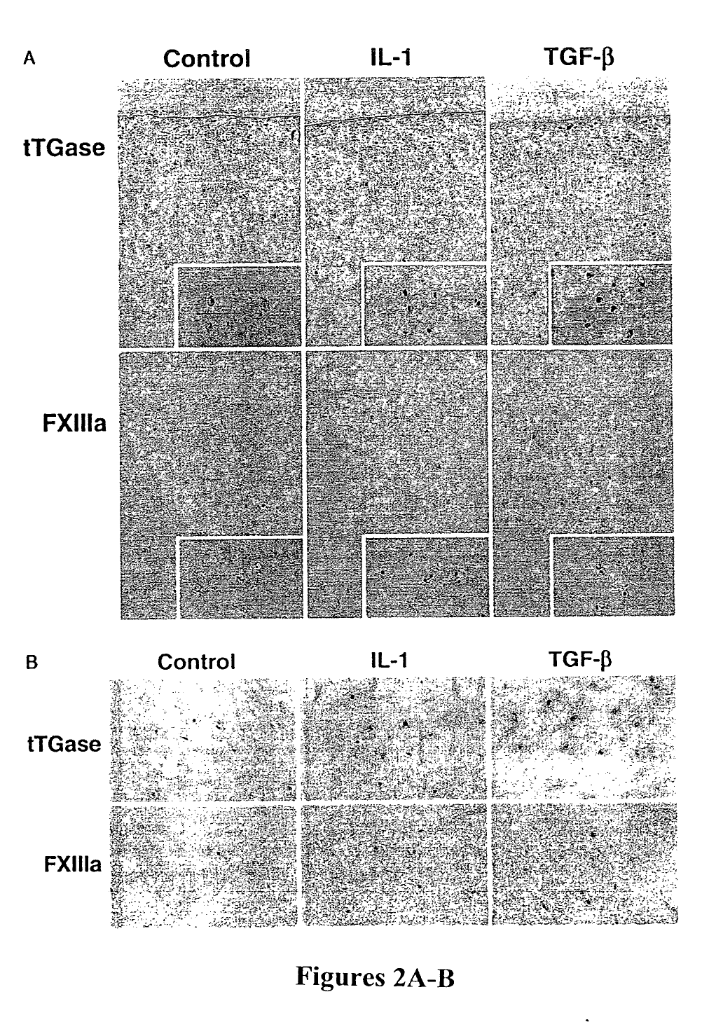

Because expression of tTGase and Factor XIIIa was increased in OA, we next studied the effects on TGase expression of TGFβ and IL-1β, both of whose activities are up-regulated in OA (42). Both TGFP (10 ng/ml) and IL-1β(1 ng/ml) induced increased tTGase and Factor XIIIa immunostaining in knee articular cartilage (FIG. 2A) and meniscal cartilage slices (FIG. 2B) carried in organ culture for 48 hours. To better understand the potential functional implications of the TGase expression detected in joint cartilages, we proceeded to study regulation of enzyme activity of TGases in knee menisci and and cultured meniscal cells.

TGase Activity in Menisci and Meniscal Cells

Knee meniscal TGase activity increased in a donor age-dependent manner in whole tissue extracts from a panel of meniscal specimens from adult donors (FIG. 3A). As additional controls, we assessed and compared the activities of other types of matrix calcification-regulatory enzymes in menisci from these donors. Thus, we studied PPi-generating NTPPPH activity, because it rises significantly in association with both aging and chondrocalcinosis in articular cartilages, and is inducible by TGFβ in chondrocytes (1,28). In addition, we studied PPi-degrading alkaline phosphatase (AP) activity, because it does not rise in aging cartilages (1). NTPPPH activity but not (AP) activity (FIG. 3B) increased in an age-dependent manner in the same panel of specimens in which TGase activity was augmented.

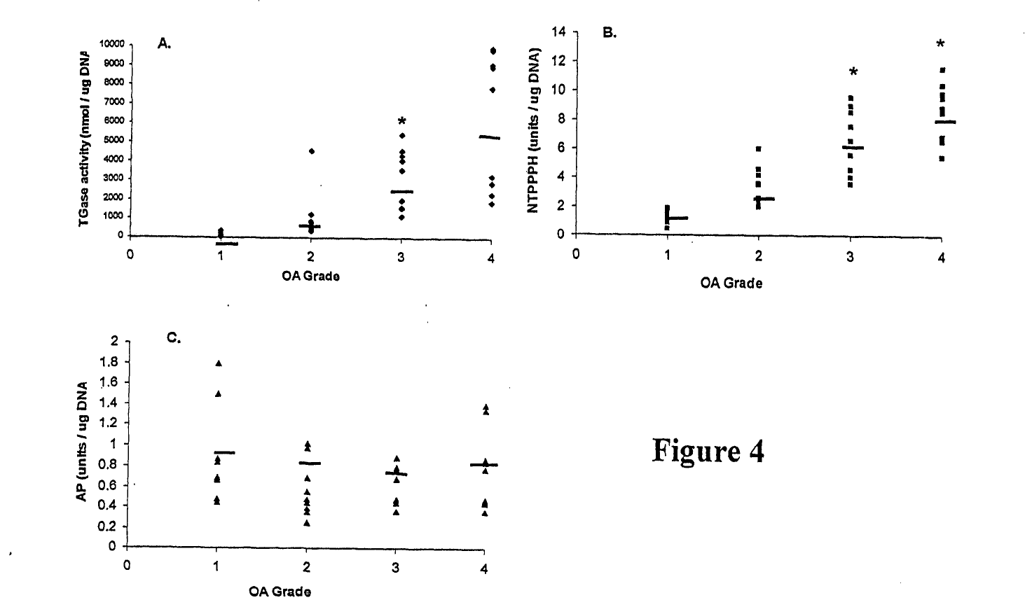

We next examined the potential relationship between OA severity grade and TGase activity in a separate group of donors over age 60, whose samples were graded for the degree of OA. We observed a direct correlation between the grade of OA and mean specific activity of TGase (per μg DNA) (FIG. 4A). There also was a significant direct correlation between the severity of OA and the specific activity of NTPPPH in knee meniscal specimens, but, in contrast, there was no significant correlation between the grade of OA and AP activity (FIG. 4B-C).

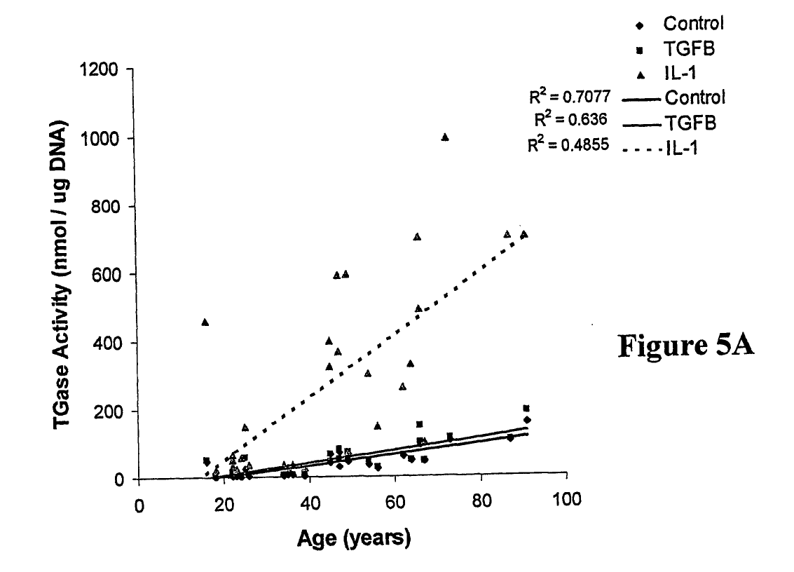

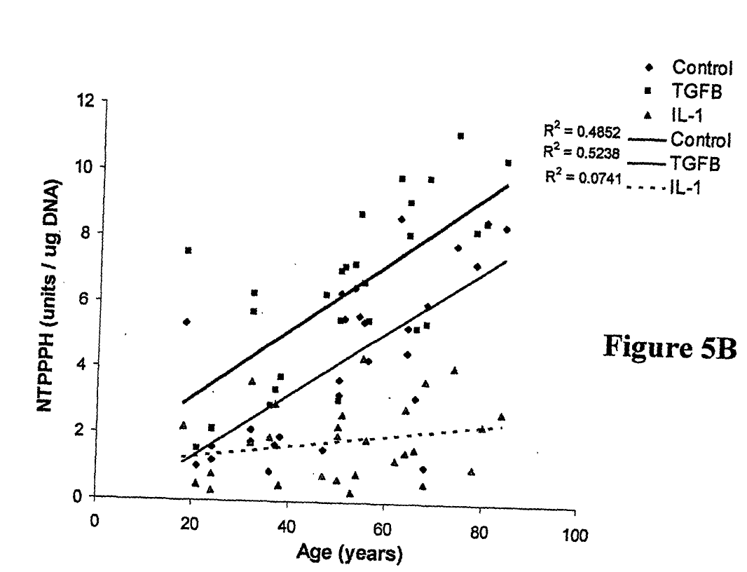

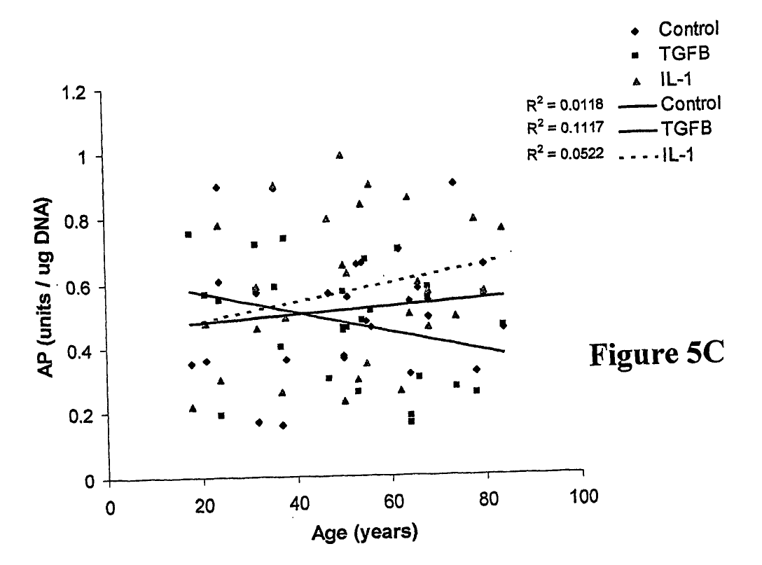

Because TGase activity increased in association with OA severity, we studied the effects on meniscal cell TGase activity in vitro of two putative mediators of OA (IL-1 and TGFβ (42). IL-1β (10 ng/ml) induced increased TGase activity in a donor age-dependent manner (and did so to a much greater degree than TGFβ (10 ng/ml) in cultured normal meniscal cartilage cells (FIG. 5A). In contrast, TGF β but not IL-1 stimulated increased NTPPPH activity in association with aging (FIG. 5B), and AP activity did not significantly change in response to either IL-1 or TGFβ or alter with aging (FIG. 5C).

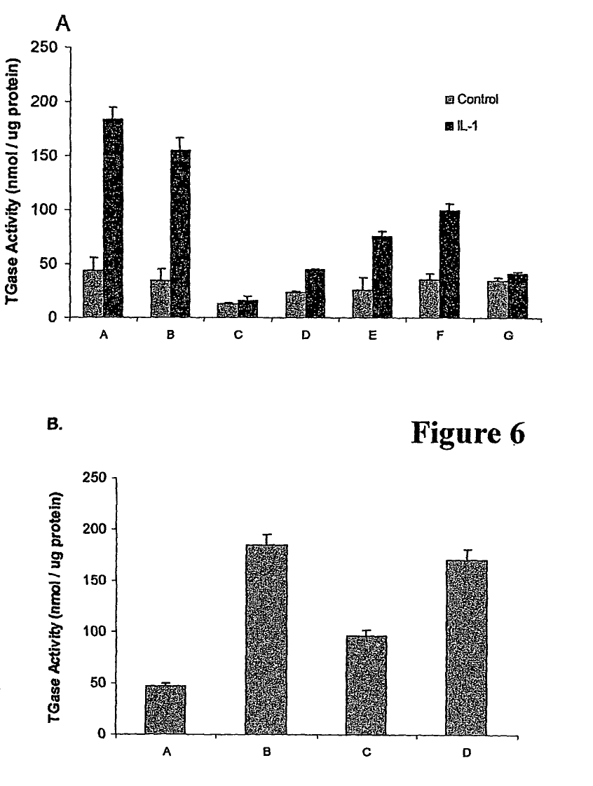

We performed immunoprecipitation studies to determine if IL-1 induced increased TGase activity attributable to each TGase. Antibodies to both Factor XIIIa and tTGase (but not nonimmune control IgG) removed TGase activity from cell lysates of IL-1-stimulated meniscal cells (FIG. 6A). Though the antibodies to Factor XIIIa and tTGase both significantly cleared TGase activity from the lysates of IL-1 -stimulated meniscal cells, there was differential recovery of TGase activity in the washed immunoprecipitates (FIG. 6A), associated with greater neutralizing activity for TGase of the antibody to Factor XIIIa than the antibody to tTGase (FIG. 6B).

Mechanism of induction of TGase activity by IL-1

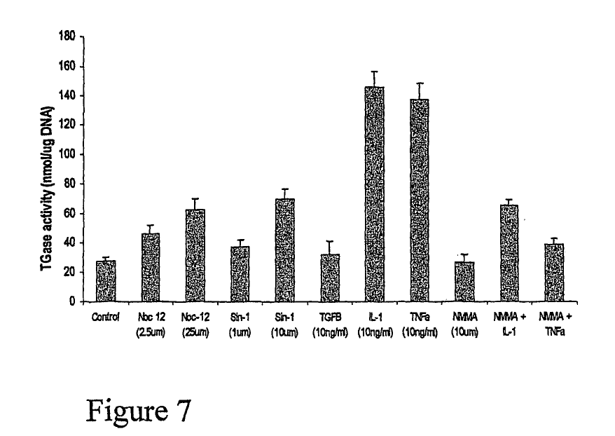

IL-1 -induced NO generation mediates certain IL-1 effects in chondrocytes (43). We observed that the NO donor Noc-12 (2.5-25 μM), and the peroxynitrite generator Sin-1(1-10 μM) (44) shared the ability of II-1 to induce TGase activity in cultured normal knee meniscal cells (FIG. 7). TNFα, which also acts on chondrocytes (45,46), stimulated increased TGase activity in cultured normal knee meniscal cells (FIG. 7). In contrast, TGFβ did not induce TGase activity under these conditions (FIG. 7). The NOS inhibitor NMMA blocked the ability of both IL-1 and TNFα to induce TGase activity (FIG. 7). Thus, we further investigated the mechanism of induction of TGase activity.

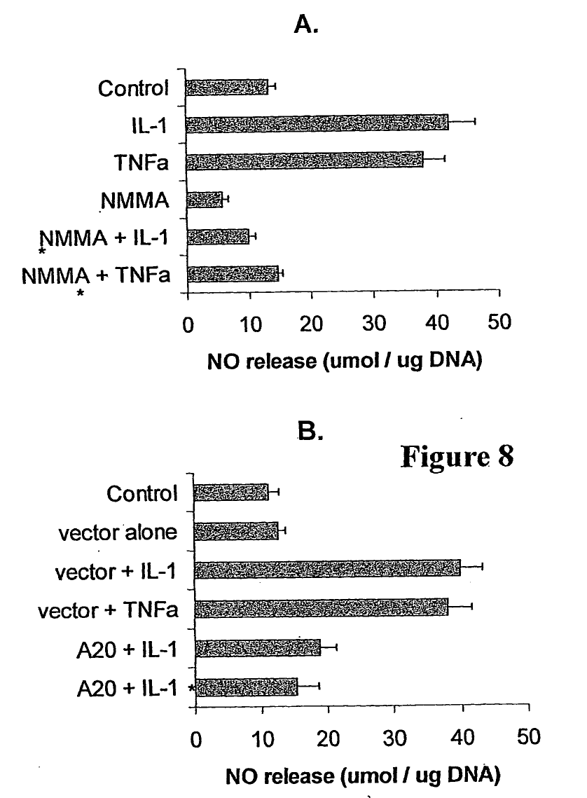

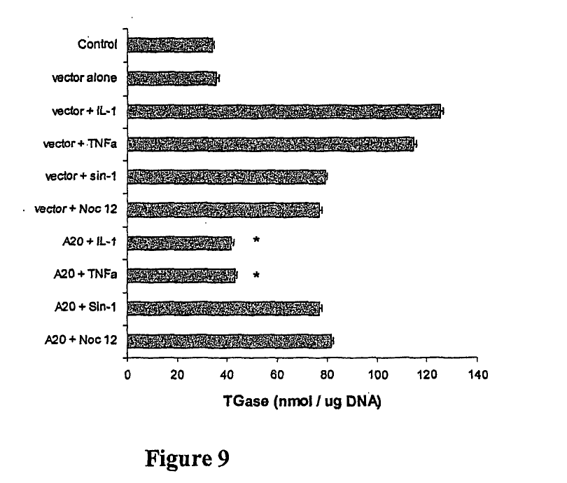

IL-1 and TNFα signaling both transduce signaling through TNFα receptor-associated signaling factors (TRAFs), TRAF2 and TRAF6 (47-50). The widely expressed zinc finger protein A20 inhibits both IL-1 and TNFβα signaling partly at the level of TRAF2 and TRAF6 action. (36,47,48,51,52), and A20 can suppress IL-1 induced NO production (52). Resting meniscal cells in culture had weak or undetectable A20 expression, but when we employed a plasmid DNA transfection approach, as described in the Methods, to efficiently express recombinant human A20 in cultured meniscal cells, we confirmed that transfection markedly up-regulated meniscal cell production of A20 as a 72 kDa polypeptide by Western blotting (not shown). Under these conditions, A20, like NMMA, attenuated IL-1 and TNFα-induced NO release (FIG. 8), and A20 (FIG. 9), like NMMA (FIG. 7) attenuated LL-1 and TNFα-induced TGase activity (FIG. 9). However, A20 did not inhibit TGase activity induced by direct provision of the NO donor Noc-12 or the peroxynitrite donor Sin-1 (FIG. 9).

Direct Effects of Factor XIIIa and tTGase on Matrix Calcification

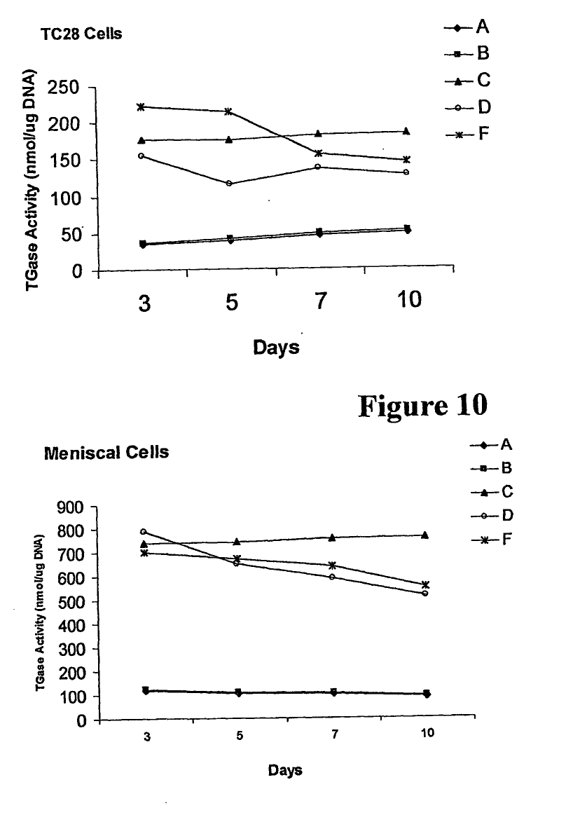

Last, we evaluated and compared the direct functional effects of Factor XIIIa and tTGase in cultured meniscal cells. Because human articular chondrocytes are difficult to transfect efficiently, we also transfected TC28 cells (28), an immortalized line of human juvenile costal chondrocytes that we confirmed to express collagen II and aggrecan (not shown). We studied cells in a system where matrix calcification was promoted in nodule-forming nonadherent chondrocytes in short-term culture (37) by the use of polyHEME—coated tissue culture plates and media supplemented with dexamethasone (10⁻⁸M), the phosphate source β-glycerophosphate, and ascorbate (50 μg/ml) (39). Transfection of both Factor XIIIa and tTGase markedly increased TGase activity in both cultured knee meniscal cells and TC28 cells (FIG. 10). Treatment with IL-1 induced significant increases in TGase activity under these conditions (FIG. 10). TGF-βdid not significantly augment activity of TGase in chondrocytes cultured in this manner.

Chondrocyte apoptosis appears to be pro-mineralizing in vitro and in vivo in growth plate and articular cartilages (4,53), and increased TGase expression can induce and modulate apoptosis in cultured cells (20-23,54,55). However, increased activity directly associated with transfection of each TGase was not associated with increased meniscal cell or TC28 cell apoptosis, measured by TUNEL assay as well as by caspase-1 and caspase-3 activation (not shown).

Extracellular PPi is a major regulator of matrix calcification (1,28), and certain pharmacologic TGase inhibitors concomitantly suppressed TGase activity and chondrocyte extracellular PPi levels in a recent study (27). However, significantly augmented TGase activity, induced by either Factor XIIIa or tTGase, failed to induce significant changes in extracellular PPi, or in PPi-regulating NTPPPH activity or AP activity (not shown).

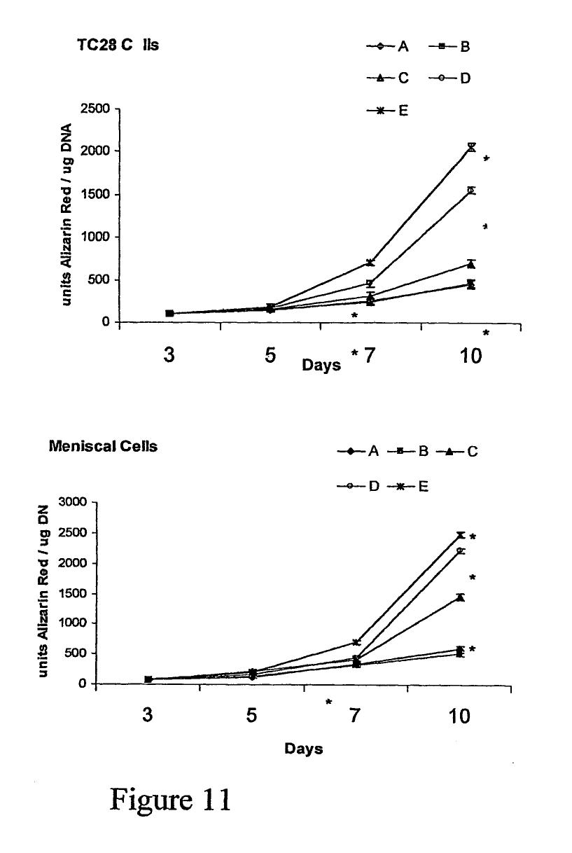

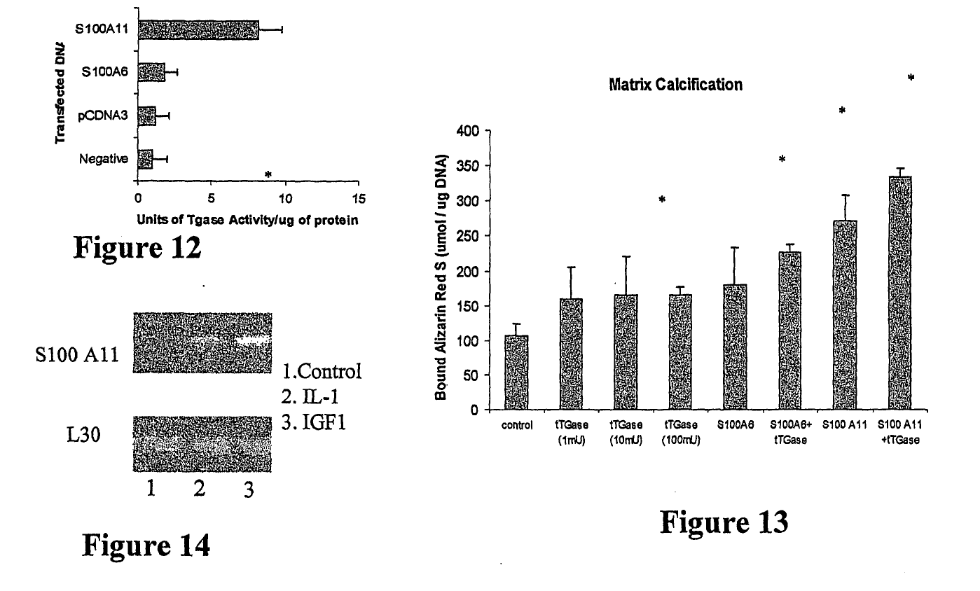

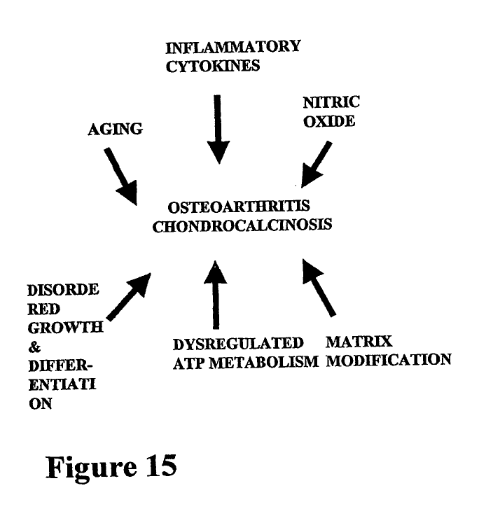

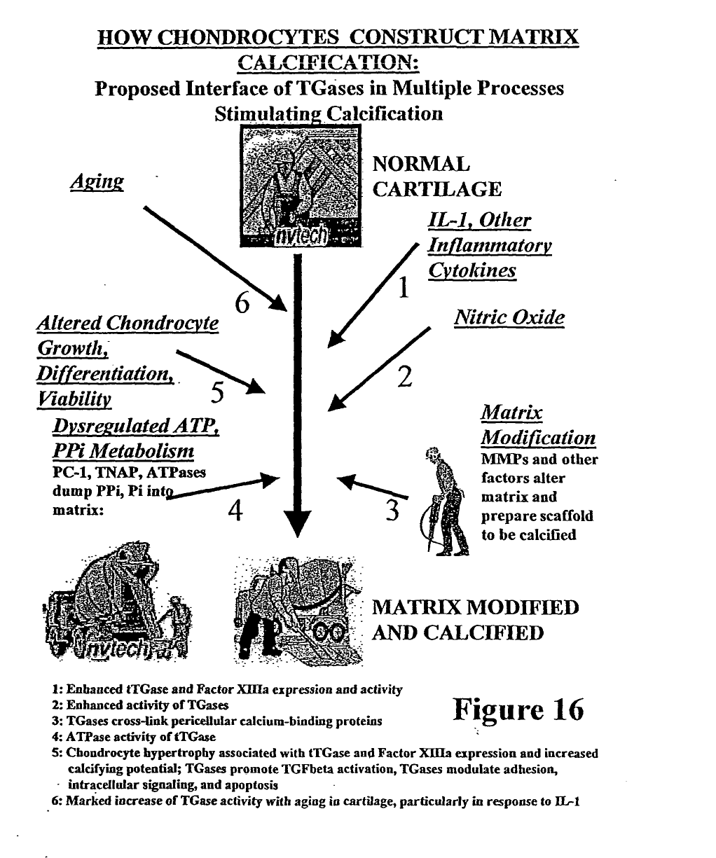

Finally, we assessed calcification of the pericellular matrix of cultured meniscal cells and TC28 cells in the same culture system, using a quantitative Alizarin red binding assay. TEM and electron diffraction analysis of the type of crystals deposited in this system, revealed, under all conditions, exclusively spherulitic HA, with D-spacings of the observed crystals on electron diffraction of 3.44,3.08,2.81,2.78, and 2.63 Angstroms. Under these conditions, L-1 but not TGF-P significantly increased matrix calcification (FIG. 11). Direct increases in both Factor XIIIa and tTGase induced particularly marked increases in the amount of calcium precipitated in the matrix (FIG. 11).

### Discussion

In this study, we demonstrated up-regulated expression of two TGases, Factor XIIIa and tTGase, in knee cartilages with OA. The levels of TGase activity in tissues are modulated by both gene expression and by a variety of post-translational events that regulate enzymatic activity of the translated TGases (12). Thus, we focused on the potential relationships between OA, aging, and TGase activity, and directly explored for direct regulatory functions of activated Factor XIIIa and tTGase in cartilage matrix calcification. Our results directly linked increased activity of TGase to aging, to increasing severity of OA of the knee, and to cartilage matrix calcification. Our findings also established the potential for joint inflammation to increase both cartilage TGase activity and matrix calcification.

In specimens from knee OA, up-regulated chondrocyte expression of Factor XIIIa and tTGase was observed in the superficial and deep zones of articular cartilages, as well as the central zones of knee menisci, in association with cells that were grossly enlarged in size. Up-regulated chondrocyte expression of Factor XIIIa and tTGase in the hypertrophic zone of growth plate cartilage (11) was confirmed in control specimens in this study. It will be of interest to further examine the direct relationship between specific markers of chondrocyte hypertrophy, or chondrocyte apoptosis, and the expression of individual TGases in situ in knee meniscal or articular cartilage specimens.

In the avian skeleton, chondrocyte hypertrophy has been linked to the ability to convert latent Factor XIIIa to an active TGase (10). Here, we observed that normal, cultured human knee meniscal chondrocytic (collagen II and aggrecan-expressing) cells transfected with tTGase or with the Factor XIIIa zymogen developed marked increases in TGase activity. In a previous study of chick sternal chondrocytes, non-hypertrophic cells did not effectively convert transfected latent Factor XIIIa to an active TGase (10). In this study, activation of Factor XIIIa TGase after transfection might have been attributable in part to cell stress from our transfection approach. Alternatively, human meniscal and articular chondrocytic cells may have a different capacity than chick sternal chondrocytes to activate latent Factor XIIIa TGase activity.

We observed that two putative mediatiors of OA, TGFβ and IL-1 (42,45), induced Factor XIIIa and tTGase expression in articular cartilages affected by OA. But TGFβ, unlike IL-1, did not induce increased TGase activity in cultured meniscal cells. In this context, TGases in chondrocytes and other cells have the capacity to promote the activation of TGFβ from the latent form (12,19). Moreover, TGFβ expression increases in both the superficial and deep zones of articular cartilages in OA (56). Thus, activation of Factor XIIIa and/or tTGase activation by inflammatory stimuli could modulate TGFβ activation in OA cartilage. Because TGFβ induces chondrocyte expression of the matrix metalloproteinase (MMP) MMP-13 (56), the activation of Factor XIIIa and tTGase also could modulate cartilage matrix degradation in OA through this TGFβ-mediated pathway.

We determined that IL-1 induced cellular TGase activity in a manner mediated by NO production. We also demonstrated that NO donors, and TNFβ increased chondrocytic cell TGase activity. Assessment of the TGase activity induced by IL-1 in cultured meniscal cells, using TGase-selective antibodies, identified contributions of both Factor XIIIa and tTGase to the increased TGase activity. Possible factors in NO-induced, IL-1 -induced, and TNFα-induced increases in TGase activity would be anticipated to include post-translational TGase phosphorylation, fatty acylation, and proteolytic cleavage (12). Tissue forms of TGases are primarily cytosolic, but tTGase can concentrate in specialized areas on the inner leaflet of the plasma membrane (12). In addition, tTGase and factor XIIIa can be partly extruded from cells (10,12), and tTGase co-localalization with pericellular fibronectin could modulate matrix assembly (57). Thus, potential regulatory effects of alterations of TGase structure on TGase subcellular localization also will be of interest to investigate as potential modulators of TGase activity and functions in chondrocytes.

IL-1 markedly induced TGase activity in chondrocytic cells, yet IL-1 treatment appeared to have a lesser enhancing effect on matrix calcification than did direct expression of Factor XIIIa and tTGase in this study. Preparation of the pericellular matrix for mineral deposition involves modulation of expression, synthesis and degradation of the collagenous and noncollagenous matrix constituents. Therefore, it is possible that catabolic effects of IL-1 for matrix protein synthesis and degradation (42) imposed limits on the extent of any increases in matrix calcification attributable to TGase activity.

In this study, we observed that IL-1-induced TGase activity was under the regulatory control of the widely expressed cytosolic zinc finger protein A20 (47-49,51,52). A20 acts to limit apoptosis and the NF-κB-mediated expression of genes including iNOS in vitro and in vivo (52). Though A20 is a broader inhibitor of TNFα than IL-1 responsiveness (51), A20 does suppress IL-1 induced NO production in cultured pancreatic beta cells (52), similar to our findings in chondrocytic cells in this study. A20 inhibits TRAF2 and TRAF6 signaling pathways employed by both TNFα and IL-1 receptors, but A20 also interacts with other cytokine-inducible signaling pathways that mediate NF-κB activation (47-50). Thus, it is possible that the ability of A20 to attenuate IL-1- induced TGase activity may have been mediated via effects that extended beyond suppression of IL-1 -induced NO production.

Constitutive A20 expression is generally low (51,52), a finding reiterated in cultured meniscal cells in this study. However, A20 is induced by a variety of cytokines and cell stressors (including IL-1, LPS, and CD40/CD40L ligation, and the Tax protein of HIV-1) in a manner mediated in part by two NF-κB binding sites in the A20 promoter (52,58). It will be of interest to determine if cartilage A20 expression is functionally altered in vivo in degenerative joint disease, cartilage aging and chondrocalcinosis, and whether targeted regulation of A20 can affect cartilage degradation and matrix calcification in vivo.

TGases have long been postulated to directly promote skeletal matrix calcification, in part by cross-linking calcium binding proteins in the pericellular matrix (12,14,23,26). TGase activity increases in association with aging in porcine chondrocytes and in chondrocyte-derived matrix vesicles, which have the potential to induce cartilage “seeding” with calcium containing crystals (27). Increased generation by chondrocytes of PPi also is associated with aging cartilage (27). Moreover, “loss of function” of TGase induced by pharmacologic inhibitors is associated with decreased extracellular levels of PPi in chondrocytes (27). But we observed no significant effects of increased TGase activity on extracellular levels of PPi or on PPi-generating NTPPPH activity. Moreover, despite the potential for direct expression of TGases to promote apoptosis in cultured cells (20), we did not observe significant induction of chondrocytic cell apoptosis, which promotes chondrocyte matrix calcification in vivo and in vitro (4,53). Nevertheless, “gain of function” of TGase activity via direct expression of Factor XIIIa and tTGase directly promoted matrix calcification by meniscal cells and chondrocytic TC28 cells in vitro.

In further studies of cultured meniscal cells, matrix vesicle TGase specific activity has not significantly increased in response to transfection of either factor XIIIa or tTGase, and matrix vesicles derived from cells transfected with these TGases did not have an elevated ability to precipitate more calcium in vitro (Johnson, K. et al, unpublished observations). Thus, the capacity of elevated TGase activity to promote matrix calcification observed in this study is not likely to be attributable to altered functional properties of matrix vesicles, changes in PPi metabolism or apoptosis. Extracellular TGase activity promotes polymerization of secreted calcium-binding proteins, such as S-100 and osteonectin (14,15,59), which could promote extracellular calcium precipitation. Thus, we speculate that effects of elevated TGase activities to stabilize pericellular calcium-binding proteins (12) promoted matrix calcification in this study, but that intracellular TGase activities also may have been at play. Specifically, increased intracellular TGase activity can affect signal transduction (12-14,16) and promote extrusion of cytosolic contents (including TGases) in chondrocytes (10).

Interestingly, tTGase exerts several unique intracellular regulatory effects on signal transduction (12,16), yet Factor XIIIa and tTGase activities similarly promoted matrix calcification. This finding, and the co-localization of Factor XIIIa and tTGase in both growth plate and OA cartilage specimens argues for a potentially redundant, central mechanism for regulation of cartilage matrix calcification. The absence of clinically defined bone or joint pathology in Factor XIIIa-deficient humans lends further support to this notion (25,60).

In conclusion, the results of this study established direct ties between increased IL-1 and TNFα expression, increased NO production, dysregulated TGase activity, and the assembly of a chondrocyte pericellular matrix that supports pathologic calcification, particularly in aging joint cartilages. Cartilage Factor XIIIa and tTGase appear to be molecular targets for the control of cartilage matrix calcitication.

### References

1. Ryan L M, McCarty D J: Calcium pyrophosphate crystal deposition disease, pseudogout, and articular chondrocalcinosis. Arthritis and Allied conditions: A textbook of Rheumatology, Edited by Koopman W. 13th Edition, Baltimore, Williams and Wilkins; 1997, pp. 2103-2126

2. Halverson P B, McCarty D: Basic calcium phosphate (apatite, octacalcium phosphate, tricalcium phosphate) crystal deposition diseases; calcinosis. Arthritis and Allied conditions: A textbook of Rheumatology, Edited by Koopman W. 13th Edition, Baltimore, Williams and Wilkins; 1997, pp. 2127-2146

3. Stevens D A, Williams G R: Hormone regulation of chondrocyte differentiation and endochondral bone formation. Molec Cell Endocrinol 1999, 151:195-204

4. Kirsch T, Swoboda B, Nah H: Activation of annexin II and V expression, terminal differentiation, mineralization and apoptosis in human osteoarthritic cartilage. Osteoarthritis Cartilage 2000, 8:294-302

5. Goomer R, Johnson K, Burton D, Amiel D, Maris T, Gujral A, Deftos L J, Terkeltaub R A: Tetrabasic C-Terminal Motif Determines Intracrine Regulatory Effects of PTHrP 1-173 on PPi Metabolism and Collagen Synthesis in Chondrocytes. In Press, Endocrinology

6. Terkeltaub R, Lotz M, Johnson K, Deng D, Hashimoto S, Goldring M B, Burton D, Deftos L J: Parathyroid hormone-related protein is abundant in osteoarthritic cartilage, and the parathyroid hormone-related protein 1-173 isofom is selectively induced transforming growth factor beta in articular chondrocytes and suppresses generation of extracellular inorganic pyrophosphate. Arthritis Rheum 1998, 41:2152-2164

7. Eerola I, Salminen H, Lammi P, Lammi M, von der Mark K, Vuorio E, Saamanen, AM: Type X collagen, a natural component of mouse articular cartilage: association with growth, aging, and osteoarthritis. Arthritis Rheum 1998, 41:1287-1295

8. Hashimoto S, Takahashi K, Ochs R L, Coutts R D, Amiel D, Lotz M: Nitric oxide production and apoptosis in cells of the meniscus during experimental osteoarthritis. Arthritis Rheum 1999, 42:2123-2131

9. Ishikawa K, Masuda I, Ohira T, Yokoyama M: A histological study of calcium pyrophosphate dihydrate crystal-deposition disease. J. Bone Joint Surg Am Vol 1989, 71:875-886

10. Nurmiskaya M, Magee C, Nurminsky D, Linsenmayer T F: Plasma transglutaminase in hypertrophic chondrocytes: Expression and cell-specific intracellular activation produce cell death and externalization. J. Cell Biol 1998, 142:1135-1144

11. Linsenmayer T F, Long F, Nurminskaya M, Chen Q, Schmid T M: Type X collagen and other up-regulated components of the avian hypertrophic cartilage program. Prog Nuc Acid Res Molec Biol 1998, 60:79-109

12. Aeschlimann D, Thomazy V: Protein crosslinking in assembly and remodelling of extracellular matrices: the role of transglutaminases. Conn Tiss Res 2000, 41:1-27

13. Greenberg C S, Birckbichler P J, Rice R H: Transglutaminases: Multifunctional crosslinking enzymes that stabilize tissues. FASEB J. 1991, 5:3071-3078

14. Aeschlimann D, Mosher D, Paulsson M: Tissue transglutaminase and factor XIII in cartilage and bone remodeling. Semin Thromb Haemostas 1996, 22:437-443

15. Aeschlimann D, Kaupp O, Paulsson M: Transglutaminase-catalyzed matrix cross-linking in differentiating cartilage: identification of osteonectin as a major glutaminyl substrate. J. Cell Biol 1995, 129:881-892

16. Chen J S, Mehta K: Tissue transglutaminase: an enzyme with a split personality. Int J. Biochem Cell Biol, 1999, 31:817-836

17. Takahashi H, Isobe T, Horibe S, Takagi J, Yokosaki Y, Sheppard D, Saito Y: Tissue transglutaminase, coagulation factor XIII, and the pro-polypeptide of von Willebrand factor are all ligands for the integrins alpha 9beta 1 and alpha 4beta 1. J. Biol Chem 2000, 275:23589-23595

18. Akimov S S, Krylov D, Fleischman L F, Belkin A M: Tissue transglutaminase is an integrin-binding adhesion coreceptor for fibronectin. J. Cell Biol 2000, 148:825-838

19. Rosenthal A K, Gohr C M, Henry L A, Le M: Participation of transglutaminase in the activation of latent transforming growth factor betal in aging articular cartilage. Arthritis Rheum 2000, 43:1729-1733

20. Melino G, Piacentini M: ‘Tissue’ transglutaminase in cell death: a downstream or a multifunctional upstream effector? Febs Lett 1998, 430:59-63

21. Oliverio S, Amendola A, Rodolfo C, Spinedi A, Piacentini M: Inhibition of “tissue” transglutaminase increases cell survival by preventing apoptosis. J. Biol Chem 1999, 274:34123-34128

22. Fabbi M, Marimpietri D, Martini S, Brancolini C, Amoresano A, Scaloni A, Bargellesi A, Cosulich E: Tissue transglutaminase is a caspase substrate during apoptosis. Cleavage causes loss of transamidating function and is a biochemical marker of caspase 3 activation. Cell Death Diff 1999, 6:992-1001

23. Thomazy V A, Davies P J: Expression of tissue transglutaminase in the developing chicken limb is associated both with apoptosis and endochcndral ossification. Cell Death Diff 1999, 6:146-154

24. Rittmaster R S, Thomas L N, Wright A S, Murray S K, Carlson K, Douglas R C, Yung J, Messieh M, Bell D, Lazier C B: The utility of tissue transglutaminase as a marker of apoptosis during treatment and progression of prostate cancer. J. Urology 1999, 162:2165-2169

25. Muszbek L, Yee V C, Hevessy Z: Blood coagulation factor XIII: structure and function. Thromb Res 1999, 94:271-305

26. Aeschlimann D, Wettenwald A, Fleisch H, Paulsson M: Expression of tissue transglutaminase in skeletal tissues correlates with events of terminal differentiation in chondrocytes. J. Cell Biol 1993, 120:1461-1470

27. Rosenthal A K, Derfus B A, Henry L A: Transglutaminase activity in aging articular chondrocytes and articular cartilage vesicles. Arthritis Rheum 1997, 40:966-970

28. Johnson K, Vaingankar S, Chen Y, Moffa A, Goldring M B, Sano K, Jin- Hua P, Sali A, Goding J, Terkeltaub R: Differential mechanisms of PPi production by plasma cell membrane glycoprotein-1 (PC-1) and B10 in chondrocytes. Arthritis Rheum 1999, 42:1986-1997

29. Vincenz C, Dixit VM: 14-3-3 proteins associate with A20 in an isoform-specific manner and function both as chaperone and adapter molecules. J. Biol Chem 1996, 271:20029-20034

30. Hashimoto S, Ochs R L, Komiya S, Lotz M: Linkage of chondrocyte apptosis and cartilage degradation in human osteoarthritis. Arthritis Rheum 1998, 41:1632-1638

31. Messner K, Gao J: The menisci of the knee joint. Anatomical and functional characteristics, and a rationale for clinical treatment. J. Anat 1998, 193:161-178

32. Yoshioka M, Shimizu C, Harwood F L, Coutts R D, Amiel D: The effects of hyaluronan during the development of osteoarthritis. Osteoarthritis Cartilage 1997, 5:251

33. Hashimoto S, Takahashi K, Ochs R L, Coutts R D, Amiel D, Lotz M: Nitric oxide production and apoptosis in cells of the meniscus during experimental osteoarthritis. Arthritis Rheum 1999, 42:2123-2131

34. Hashimoto S, Setareh M, Ochs R L, Lotz M: Fas/Fas ligand expression and induction of apoptosis in chondrocytes. Arthritis Rheum 1997, 40:1749-1755

35. Hettasch J M, Peoples K A, Greenberg C S: Analysis of factor XIII substrate specificity using recombinant human factor XIII and tissue transglutaminase chimeras. J Biol Chem 1997, 272:25149-25156

36. Jaattela M, Mouritzen H, Elling F, Bastholm L: A20 zinc finger protein inhibits TNF and IL-1 signaling. J. Immunol 1996, 156:1166-1173

37. Reginato A M, lozzo R V, Jimenez S A: Formation of nodular structures resembling mature articular cartilage in long-term primary cultures of human fetal epiphyseal chondrocytes on a hydrogel substrate. Arthritis Rheum 1994, 37:1338-1349

38. Bodine P V, Vernon S K, Komm B S: Establishment and hormonal regulation of a conditionally transformed preosteocytic cell line from adult human bone. Endocrinol 1996, 137:4592-4604

39. Johnson K, Moffa A, Pritzker K, Chen Y, Goding J, Terkeltaub R: Matrix vesicle Plasma Cell Membrane Glycoprotein-1 (PC-1) regulates mineralization by murine osteoblastic MC3T3 cells. J. Bone Min Res 1999, 14:883-892

40. Cheng P T, Pritzker K P: The effect of calcium and magnesium ions on calcium pyrophosphate crystal formation in aqueous solutions. J. Rheumatol 1981, 8:772-782

41. Cheng P T, Pritzker K P, Kandel R A, Reid A: Analytical scanning and transmission electron microscopy and x-ray microdifiactometry of calcium pyrophosphate dihydrate crystal deposits in tissues. Scan Electron Microscopy 1983, (Pt 1): 369-377

42. Goldring M B: The role of the chondrocyte in osteoarthritis. Arthritis Rheum 2000, 43:1916-1926

43. Studer R, Jaffurs D, Stefanovic-Racic M, Robbins P D, Evans C H: Nitric oxide in osteoarthritis. Osteoarthritis Cart 1999, 7:377-379

44. Johnson K, Jung A, Murphy A, Andreyev A, Dykens J, Terkeltaub R: Mitochondria I oxidative phosphorylation is a downstream regulator of nitric oxide effects on chondrocyte matrix synthesis and mineralization. Arthritis Rheum 2000, 43:1560-1570

45. Geng Y, Valbracht J, Lotz M: Selective activation of the mitogen-activated protein kinase subgroups cJun NH2 terminal kinase and p38 by IL-1 and TNF in human articular chondrocytes. J. Clin Invest 1996, 98:2425-2430

46. Westacott C I, Barakat A F, Wood L, Perry M J, Nelson P, Bisbinas I, Armstrong L, Millar A B, Elson C J: Tumor necrosis factor alpha can contribute to focal loss of cartilage in osteoarthritis. Osteoarthritis Cart 2000, 8:213-221

47. Heyninck K, De Valck D, Vanden Berghe W, Van Criekinge W, Contreras R, Fiers W, Haegeman G, Beyaert R: The zinc finger protein A20 inhibits TNF-induced NF-kappaB-dependent gene expression by interfering with an RIP- or TRAF2-mediated transactivation signal and directly binds to a novel NF-kappaB-inhibiting protein ABIN. J Cell Biol 1999, 145:1471-1482

48. Heyninck K, Beyaert R: The cytokine-inducible zinc finger protein A20 inhibits IL-1 -induced NF-kappaB activation at the level of TPAF6. Febs Letters 1999, 442:147-150

49. Jobin C, Holt L, Bradham C A, Streetz K, Brenner D A, Sartor R B: TNF receptor-associated factor-2 is involved in both IL-1 beta and TNF-alpha signaling cascades leading to NF-kappa B activation and IL-8 expression in human intestinal epithelial cells. J. Immunol 1999, 162:4447-4454

50. Malinin N L, Boldin M P, Kovalenko A V, Wallach D: MAP3K-related kinase involved in NF-kappaB induction by TNF, CD95 and IL-1. Nature 1997, 385:540-544

51. Lee E G, Boone D L, Chai S, Libby S L, Chien M, Lodolce J P, Ma A: Failure to regulate TNF-induced NF-kappaB and cell death responses in A20-deficient mice. Science 2000, 289:2350-2354

52. Grey S T, Arvelo M B, Hasenkamp W, Bach F H, Ferran C: A20 inhibits cytokine-induced apoptosis and nuclear factor kappaB-dependent gene activation in islets. J. Exp Med 1999, 190:1135-1146

53. Hashimoto S , Ochs R L, Rosen F, Quach J, McCabe G, Solan J, Seegmiller J E, Terkeltaub R, Lotz M: Chondrocyte-derived apoptotic bodies and calcification of articular cartilage. Proc Natl Acad Sci USA 1998, 95:3094-3099

54. Piredda L, Farrace M G, Lo Bello M, Malomi W, Melino G, Petruzzelli R, Piacentini M: Identification of ‘tissue’ transglutaminase binding proteins in neural cells committed to apoptosis. FASEB J. 1999, 13:355-364

55. Fesus L: Transglutaminase-catalyzed protein cross-linking in the molecular program of apoptosis and its relationship to neuronal processes. Cell Molec Neurobiol 1998, 18:683-694

56. Moldovan F, Pelletier J P, Mineau F, Dupuis M, Cloutier J M, Martel- Pelletier J: Modulation of collagenase 3 in human osteoarthritic cartilage by activation of extracellular transforming growth factor beta: role of furin convertase. Arthritis Rheum 2000, 43:2100-2109

57. Gaudry C A, Verderio E, Aeschlimann D, Cox A, Smith C, Griffin M: Cell surface localization of tissue transglutaminase is dependent on a fibronectin-binding site in its N-terminal beta-sandwich domain. J. Biol Chem 1999, 274:30707-30714

58. Krikos A, Laherty C D, Dixit V M: Transcriptional activation of the tumor necrosis factor alpha-inducible zinc finger protein, A20, is mediated by kappa B elements. J. Biol Chem 1992, 267:17971-17976

59. Gohr C M, Rosenthal A K: S 100 in aging articular chondrocytes. Arthritis Rheum 2000, 43:S281 (Abstract)

60. Anwar R, Miloszewski K J: Factor XIII deficiency. Brit J. Haematol 1999, 107:468-484

Although the invention has been described with reference to the presently preferred embodiment, it should be understood that various modifications can be made without departing from the spirit of the invention. Accordingly, the invention is limited only by the following claims.

What is claimed is:

1. A method for suppressing pathological calcification of the meniscal and articular cartilage matrix, comprising:
inhibiting activation and/or activity of zymogen factor (FXIIIa) and tissue transglutaminase (tTGase) in chondrocytes.

2. The method according to claim 1, wherein the inhibition of activation is accomplished by blocking production of a member selected from the group consisting essentially of interleukins IL-I, IL-8, nitric oxide donor Noc-12, peroxynitrite generator Sin-1, tumor necrosis factor α (TNFα), and S100 family of proteins.

3. The method according to claim 1, wherein the inhibition of activation is accomplished by blocking TNFα receptor-associated signaling factors (TRAFs), TRAF2 and TRAF6.

4. The method according to claim 3, wherein the inhibition is accomplished by expressing A20 in chondrocytic cells.

5. A method for inhibiting TGase activity of zymogen Factor XIIIa (FXIIIa) and/or tissue transglutaminase (tTGase) in a chondrocyte, comprising contacting the chondrocyte with an effective amount of an inhibitor that inhibits tTGase and/or FXIIIa.

6. The method of claim 5, wherein the inhibitor is an inhibitor of IL-1, Noc-12, Sin1, tumor necrosis factor a (TNFα), and/or TNFα receptor-associated signaling factor (TRAFs), TRAF2 and TRAF6.

7. The method of claim 5, wherein the inhibitor is a polynucleotide that inhibits tTGase or FXIIIa expression.

8. The method of claim 5, wherein the method is performed in vitro.

9. The method of claim 5, wherein the method is performed in vivo.

10. The method of claim 9, wherein the chondrocyte is from a chondrocyte-derived cell line.

11. A method for identifying an agent that affects matrix calcification, comprising
contacting a chondrocyte in vitro with a test agent under conditions for inducing matrix calcification, wherein the chondrocyte expresses zymogen factor XIIIa (FXIIIa) and/or tissue transglutaminase (tTGase); and
determining the effect of the test agent on matrix calcification, wherein an effect on matrix calcification identifies the test agent as an agent that affects matrix calcification.

12. The method of claim 11, wherein the chondrocyte is transfected with a TGase expression vector for expressing zymogen factor FXIIIa or tTGase.

13. The method of claim 12, wherein the chondrocyte is from a chondrocyte-derived cell line.

14. The method of claim 11, wherein the conditions for inducing matrix calcification include contacting the chondrocyte with an agent that activates and/or increases activity of zymogen factor FXIIIa and/or tissue transglutaminase (tTGase), wherein the agent affects the activity of IL-1, Noc-12, Sin-1, and/or tumor necrosis factor a (TNFα).

15. The method of claim 11, wherein the test agent is a nitric oxide synthase (NOS) inhibitor.
